(19) United States
(12) Patent Application Publication
(10) Pub. No.: US 2026/0174782 A1
(43) Pub. Date: Jun. 25, 2026

(54) USE OF CATIONIC STEROIDAL ANTIMICROBIAL COMPOSITIONS TO TREAT EPIDERMOLYSIS BULLOSA

(71) Applicants: Michael C. MOORE, Chandler, AZ (US); Jace SANDERS, Provo, UT (US); Paul B. SAVAGE, Mapleton, UT (US)

(72) Inventors: Michael C. MOORE, Chandler, AZ (US); Jace SANDERS, Provo, UT (US); Paul B. SAVAGE, Mapleton, UT (US)

(73) Assignee: BRIGHAM YOUNG UNIVERSITY, Provo, UT (US)

(21) Appl. No.: 19/425,354

(22) Filed: Dec. 18, 2025

Related U.S. Application Data
(60) Provisional application No. 63/736,471, filed on Dec. 19, 2024.

(51) Int. Cl. A61K 31/56; A61K 9/00; A61K 47/10; A61K 47/12; A61P 17/02

(52) U.S. Cl. CPC A61K 31/56; A61K 9/0014; A61K 9/006; A61K 47/10; A61K 47/12; A61P 17/02

(57) ABSTRACT

The present disclosure describes methods of treating epidermolysis bullosa (EB) by topically administering a treatment composition that includes one or more cationic steroidal antimicrobial (CSA) compounds and a carrier (“CSA treatment composition”). A method of therapeutically or prophylactically treating a human patient suffering from EB includes: (1) providing a CSA treatment composition that includes one or more CSA compounds and a carrier (e.g., aqueous, cream, and/or gel carrier); (2) administering the CSA treatment composition to the skin or mucous membrane of the human subject suffering from EB; and (3) the CSA treatment composition initiating wound healing, accelerating wound healing, and/or prophylactically preventing wound formation in the human subject. Example forms of CSA treatment compositions include sprays, creams, lotions, ointments, salves, lozenges, mouthwashes, popping candies, and gels, including spray gels.

### CROSS-REFERENCE TO RELATED APPLICATION

This Application claims the benefit of U.S. Provisional Application No. 63/736,471, filed Dec. 19, 2024, which is incorporated by reference in its entirety.

### BACKGROUND

Epidermolysis bullosa (EB) is a group of rare medical conditions that result in easy blistering of the skin and mucous membranes. EB is also characterized as a rare genetic disease manifested at birth by defective or deficient anchoring fibrils, primarily in skin. EB is characterized by extreme fragility of the skin and mucous membranes, which is disfiguring and very painful. Blisters occur with minor trauma or friction and are painful. Severity can range from mild to fatal. Even the mildest friction damages the skin and mucous membranes, causing severe blistering and wound formation. The propensity of a person suffering from EB to experience constant itching causes the person to scratch the itch, which exacerbates wound formation and interferes with healing. EB wounds often become chronic, resulting in significant scarring. Those with mild cases may not develop symptoms until they start to crawl or walk. Complications may include esophageal narrowing, squamous cell skin cancer, and the need for amputations.

Infection with EB can occur at any age and can lead to high rate of mortality. The disease is life altering, resulting in the inability for patients to thrive. EB cannot be detected and is unknown until birth. The disease is often fatal, typically due to sepsis and/or from wound infection.

EB is caused by a mutation in at least one of 16 different genes. Some types are autosomal dominant while others are autosomal recessive. The underlying mechanism is a defect in attachment between or within the layers of the skin. Loss or diminished function of type VII collagen leads to weakness in the structural architecture of the dermal-epidermal junction (DEJ) and mucosal membranes. There are four main disease types: epidermolysis bullosa simplex (EBS), dystrophic epidermolysis bullosa (DEB), junctional epidermolysis bullosa (JEB), and Kindler epidermolysis bullosa (KEB), formerly known as Kindler syndrome (KS). The diagnosis is suspected based on symptoms and confirmed by skin biopsy or genetic testing.

The following is a description of the pathophysiology of EB. The human skin consists of two layers: an outermost layer, or the epidermis, and a layer underneath, or the dermis. In individuals with healthy skin, there are protein anchors between these two layers (dermo epidermal junction) that prevent the layers from moving independently from one another (shearing). In people born with EB, the two skin layers lack the protein anchors that hold them together, resulting in extremely fragile skin. Even minor mechanical friction (like rubbing or pressure) or trauma can separate the layers of the skin and form blisters and painful sores. EB sufferers manifest unremitting skin blistering that evolves into chronic wounds, inflammation, and fibrosis. People with EB have compared the severity and pain of the sores or lesions with third-degree burns. Furthermore, as a complication of chronic skin damage, people with EB have an increased risk of malignancies (cancers) of the skin. Virtually any organ lined or covered by epithelium may be injured in inherited EB. External eye, esophagus, upper airway, and genitourinary tract are the epithelial surfaced tissues that are at particular risk.

The worldwide prevalence of EB sufferers is estimated to be between 300,000 to 400,000. In the U.S. it is estimated that there are between 20,000 to 40,000 current cases (comparable to cystic fibrosis). Debra, a US support organization for EB, estimates there are 30,000 current cases in the U.S. Stanford University estimates there are there are 25,000 to 50,000 current cases in the U.S. EBMRF (EB Medical Research Foundation) estimates that as many as 100,000 Americans suffer from some form of EB. An EB orphan drug application estimates there are 14,000 to 40,000 current cases in the U.S. In the E.U., it is estimated there are between 50,000 to 80,000 current cases. Gabriella Pohln-Gubo (5th ECRD, 2010). Prevalence estimates in Northern Europe include: Northern Ireland about 44 per million of population (Covello et al. J Inv Derm, 1998); Scotland about 49 per million of population (Horn et al., Brit J Derm, 2008); and Scandinavia about 40 per million of population (Rugg et al. J Inv Derm, 2007). In Japan it is estimated there are between 5,000-15,000 current cases.

There is currently no therapy in development to treat all subtypes, namely EBS, DEB, JEB, and KEB. The frequency of EBS (simplex) is about 75%, and its symptoms include blistering on hands and feet (localized) and blistering all over the body (generalized). The frequency of DEB (dystrophic) is about 20%, and its symptoms include contraction of joints, fusion of fingers and toes, contraction of mouth membranes, narrowing of the esophagus, and the possibility of skin cancer. The frequency of JEB (junctional) is about 5%, and its symptoms include marking and damage to skin or face, internal blistering of oral tracts, extensive blistering all over body, and blistering of membranes of internal organs; severe complications can often become lethal. The frequency of KEB (Kindler) is about 1%, and its symptoms include generalized blistering on body, skin atrophy, atrophic scarring, photosensitivity, oral soft-tissue abnormalities (gingival hyperplasia). esophageal strictures, and possibility of skin cancer.

FIGS. 1A-1C are photographs that illustrate badly damaged limbs and skin of infant patients suffering from epidermolysis bullosa (EB). Because there is no cure for the condition, management involves wound care, pain control, controlling infections, nutritional support, and prevention and treatment of complications. Thus, the only current treatment option is palliative treatment, mainly bandaging, which creates a significant burden for families and caregivers. FIG. 2 is a photograph that illustrates the bandaging of a limb of an infant suffering from EB. The average monthly cost of bandages can be $5,000-$15,000, excluding incremental hospital costs. Thus, treatment goals are minimizing wounds and infections. Palliative homecare involves daily wound care, protective bandaging, and pain management, sometimes requiring opiates, which are highly addictive and to which people build up tolerance. Surgery can become necessary to repair deformities. Survivors of EB, even if they reach adulthood, have a high risk of developing fatal skin cancer.

The following is a description of currently researched treatments. Research has focused on changing the mixture of keratins produced in the skin. There are 54 known keratin genes—of which 28 belong to the type I intermediate filament genes and 26 to type II—which work as heterodimers. Many of these genes share substantial structural and functional similarity, but they are specialized to cell type and/or conditions under which they are normally produced. If the balance of production could be shifted away from the mutated, dysfunctional keratin gene toward an intact keratin gene, symptoms could be reduced. For example, sulforaphane, a compound found in broccoli, was found to reduce blistering in a mouse model to the point where affected pups could not be identified visually, when injected into pregnant mice (5 μmol/day=0.9 mg) and applied topically to newborns (1 μmol/day=0.2 mg in jojoba oil).

As of 2008, clinical research at the University of Minnesota has explored allogeneic bone marrow transplantation for RD and junctional EB, treating a two-year-old child who is one of two brothers with EB. A second transplant has also been performed on the child's older brother. A Missouri boy has also successfully undergone the transplant, as well as a 5 year old boy from Alabama. So far there have been 12 successful transplants. Another transplant is scheduled for a California baby. A clinical trial is planned for 30 subjects. However, the immune suppression that bone marrow transplantation requires causes a risk of serious infections with large scale blisters and skin erosion. Indeed, at least four people have died in the course of either preparation for or institution of bone marrow transplantation for EB, out of only a small group of patients treated so far. The mechanism of action of this therapy is unclear as hematopoietic stem cells are not thought to contribute to epithelial lineages. Rather, it is speculated that cross-correction from tissue-resident graft-derived immune cells contributes to the observed clinical benefit.

A pilot study performed in 2015 suggests that systemic granulocyte-colony stimulating factor (G-CSF) may promote increased wound healing in people with dystrophic EB. Transplanting skin derived from genetically modified stem cells onto the wound surfaces has been studied with a report of improvements in one person.

A 2017 clinical trial with male RDEB (recessive dystrophic EB) patients conducted successful grafting of type VII gene corrected keratinocytes (COL7A1 gene correction using retrovirus transduction), without any serious adverse effects. Type VII collage formation was observed at the dermis-epidermis junction in significant amounts.

A 2020 study demonstrated the safe allogenic grafting of acellular dermal matrix/scaffolds in EB patients without any observed infection or necrosis and instead noted fewer required dressing changes, promoted wound healing, pain reduction, and an overall improvement in the quality of life of the patients.

In 2022, a pharmaceutical gel made out of birch bark extract from Betula pendula and Betula pubescens was approved by the European Union as a treatment for epidermolysis bullosa.

Notwithstanding proposed treatments that remain exploratory, unproven, or of marginal therapeutic effect, there is an ongoing and severe need for compositions and methods capable of more effectively treating and preventing the debilitating symptoms associated with EB.

### SUMMARY

The present disclosure describes compositions and methods for treating epidermolysis bullosa (EB) by topically administering a treatment composition comprising one or more cationic steroidal antimicrobial (CSA) compounds and a carrier (“CSA treatment composition”) to a subject in need thereof. The treatment compositions can be applied to tissue of the subject with EB wounds or lesions or tissue of the subject that may be at risk of developing EB wounds or lesions. Example forms of CSA treatment compositions include liquids, sprays, and gels, including spray gels, that can be applied topically to therapeutically or prophylactically treat EB wounds or lesions in a subject in need thereof. The CSA treatment composition has been found to initiate wound healing, which is different than accelerating wound healing. That is because the body does not have a mechanism for healing EB wounds on its own.

In some embodiments, a method of therapeutically or prophylactically treating a human patient suffering from EB comprises: (1) providing a CSA treatment composition that includes one or more CSA compounds and a carrier (e.g., aqueous and/or gel carrier); (2) administering the CSA treatment composition to soft tissue (e.g., skin and/or mucous membrane) of the human patient that has or is at risk of having EB wounds or lesions; and (3) the CSA treatment composition initiating and/or accelerating healing of soft tissue wounds caused by EB and/or preventing or mitigating formation of lesions or soft tissue wounds caused by EB in the human patient. It will be appreciated that the CSA treatment composition can accelerate healing in tissue where wound healing has already been initiated by administering a CSA treatment composition.

In some embodiments, a method of therapeutically treating a human patient suffering from EB comprises: (1) providing a CSA treatment composition that includes one or more cationic steroidal antimicrobial (CSA) compounds and a carrier (e.g., aqueous and/or gel carrier); (2) administering the CSA treatment composition to lesions or soft tissue wounds (e.g., in the skin and/or mucous membrane) caused by EB in the human patient; and (3) the CSA treatment composition initiating and/or accelerating healing of the lesions or soft tissue wounds caused by EB in the human patient.

In some embodiments, a method of prophylactically treating a human patient suffering from EB comprises: (1) providing a CSA treatment composition that includes one or more cationic steroidal antimicrobial (CSA) compounds and a carrier (e.g., aqueous and/or gel carrier); (2) administering the CSA treatment composition to soft tissue of the human patient suffering from EB that is at risk of developing lesions or soft tissue wounds caused by EB; and (3) the CSA treatment composition preventing or mitigating formation of lesions or soft tissue wounds caused by EB in the human patient.

The carrier may be any suitable carrier in which one or more CSA compounds can be mixed. Examples include water, alcohols, DMSO, other organic solvents, oils, gels, emulsion, or combinations thereof.

In some embodiments, the treatment composition can be administered orally, such as by administering a pre-measured amount of a liquid treatment composition, which is swished around the patient's mouth (e.g., for about 10-60 seconds, or about 15-50 seconds, or about 20-40 seconds), which ensures the composition contacts all lesions in the mouth, and then swallowed to ensure the composition contacts lesions deep in the mouth and/or throat. In such cases, the treatment composition should be formulated to be safe for oral ingestion. This is still primarily topical rather than systemic administration because the composition contacts oral lesions in the mouth and throat.

The composition can be administered orally to the mouth and throat or topically to the skin or other tissue outside the mouth. When administered orally, it can be by a device that dispenses a pre-measured quantity of liquid treatment composition as a bolus or a spray (e.g., more or multiple sprays), such as about 0.1 oz (3 mL) to about 1.5 oz (44.4 mL), or about 0.25 oz (7.4 mL) to about 1.25 oz (37 mL), or about 0.4 oz (11.8 mL) to about 1 oz (29.6 mL), or about 0.5 oz (14.8 mL) to about 0.8 oz (23.7 mL).

In other embodiments, the treatment composition can be applied topically to skin or other tissue outside the mouth. In such cases, the quantity will depend on the surface area of skin or other tissue that is being treated.

In some embodiments, the treatment composition for application to skin or mucous membranes (e.g., mouth and throat) can have a concentration of the one or more CSA compounds of about 100 ppm to about 1000 ppm, or about 200 ppm to about 750 ppm, or about 300 ppm to about 500 ppm.

Any CSA compound described herein, or any combination of such CSA compounds may be utilized in a treatment composition. Presently preferred CSA compounds include CSA-43, CSA-44, CSA-45, CSA-131, CSA-142, CSA-144, CSA-145, CSA-146, CSA-148, CSA-255, CSA 4108, CSA 4110, CSA 4110R, CSA 4110S, CSA 4112, CSA 4114, CSA 4204, CSA 4206, CSA 4208, CSA 4210, and CSA 4310, structurally similar CSA compounds, or combinations thereof. These CSA compounds in particular are very similar in structure and functionality such that test data generated using treatment compositions that contain CSA-44 is an accurate and reliable proxy for treatment compositions containing other CSA compounds of similar structure and functionality (e.g., that have the same stereochemistry as CSA-44 and amine functional groups at some or all of the R₃, R₇ and R₁₂ positions).

The CSA treatment compositions have been found to be effective in providing at least three key activities, which can act both therapeutically and prophylactically to treat EB lesions and wounds. The first mechanism of action is the initiation of wound healing, which involves triggering cell migration into wound beds and promoting neovascularization. The second mechanism of action is anti-inflammatory activity, primarily by sequestering bacterial endotoxins, reducing the release of some endogenous cytokines, and up-regulating the release of IL-8 by the subject. The third mechanism of action is antimicrobial activity, including: (1) bactericidal activity against Gram-positive and Gram-negative bacteria that are or may be present, including bactericidal activity against drug-resistant bacteria and established biofilms that include bacteria; (2) fungicidal activity against pathogenic fungi that are or may be present, including fungicidal activity against drug-resistant fungi and established biofilms that include fungi; and (3) virucidal activity, including inactivating lipid-enveloped viruses that are or may be present.

Moreover, even though the disclosed CSA treatment compositions were proposed and tested because it was believed they could mimic the activity of human cathelicidin LL-37 (a cationic peptide), the CSA treatment compositions were unexpectedly and unpredictably found to far exceed the activity and efficacy of LL-37 in treating patients suffering from EB. The CSA treatment compositions have a broad impact on healing lesions and wounds caused by EB, particularly the initiation of healing of lesions and wounds caused by EB that are otherwise non-healing (i.e., they are non-healing even though LL-37 is produced by and may be present in the body of patients suffering from EB, indicating that LL-37 appears to be entirely ineffective in initiating wound healing in patients with EB).

The CSA treatment compositions have been found to signal through P2X7 and FPRL-1, generating activities observed with antimicrobial peptides, including cell migration, neovascularization and acceleration of wound healing. The CSA treatment compositions also trigger host cell migration into wound beds of EB lesions and wounds, which is unique to the tested and other disclosed CSA compounds. The CSA treatment compositions sequester endotoxins, reducing inflammation. The CSA treatment compositions stimulate angiogenesis. The CSA treatment compositions are broad spectrum antimicrobials with activity against Gram-positive and Gram-negative bacteria (including drug-resistant microbes) and fungi. The CSA treatment compositions are active against established biofilms (bacterial and fungal). Thus, the CSA treatment compositions are effective in treating patients suffering from EB that human cathelicidin LL-37 and other endogenous antimicrobial peptides are incapable of treating.

Additional features and advantages will be set forth in part in the description that follows, and in part will be obvious from the description, or may be learned by practice of the embodiments disclosed herein. It is to be understood that both the foregoing brief summary and the following detailed description are exemplary and not restrictive of the embodiments disclosed herein or as claimed.

### BRIEF DESCRIPTION OF THE DRAWINGS

In order to describe various features and concepts of the present disclosure, a more particular description of certain subject matter will be rendered by reference to specific embodiments which are illustrated in the appended drawings. Understanding that these figures depict just example embodiments and are not to be considered to be limiting in scope, various embodiments will be described and explained with additional specificity and detail through the use of the accompanying drawings in which:

FIGS. 1A-1C are photographs of badly damaged limbs of infant patients suffering from epidermolysis bullosa (EB);

FIG. 2 is a photograph of a limb of an infant patient suffering from EB that is bandaged for protection;

FIG. 3 is a diagram that schematically illustrates a cross-section of skin structure, including the different strata of skin, in a patient suffering from EB;

FIG. 4 is a diagram that schematically illustrates and compares a cross-section of healthy skin, skin of a patient suffering from the recessive form of dystrophic EB (RDEB), and a patient suffering from RDEB and with an aggressive form of squamous cell carcinoma (RDEB-cSCC);

FIG. 5 is a diagram that schematically illustrates proposed mechanisms of action by which CSA compounds can therapeutically or prophylactically treat skin of a patient suffering from EB;

FIGS. 6A and 6B are photographs of the mouth of a child suffering from EB Simplex at Day 1 and Day 4 of being treated with a treatment composition comprising a carrier and a CSA compound by oral administration and showing significant healing of EB wounds by Day 4;

FIGS. 7A and 7B are photographs of the foot of a child suffering from EB Simplex at Day 1 and Day 4 of being treated with a treatment composition comprising a carrier and a CSA compound by topical administration and showing significant healing of EB wounds by Day 4;

FIGS. 8A-9D are graphs that illustrate and compare the effects of treatment compositions comprising a carrier and a CSA compound at various concentrations on the metabolic activity of various RDEB cell lines.

FIGS. 9A-9D are graphs that illustrate and compare the effects of treatment compositions comprising a carrier and a CSA compound at various concentrations on the percent proliferation of various RDEB cell lines;

FIGS. 10A-10D are photographs of various cultured RDEB cell lines for which test data was analyzed and presented in FIGS. 8A-8D and 9A-9D;

FIG. 11 is a graph that illustrates the amounts of CSA-44 in skin tissues for when treating with different treatment composition formulations and a control.

FIG. 12A illustrates example CSA compounds having ester or amide linkages at one or more of the R₃, R₇, and R₁₂ positions;

FIG. 12B illustrates example CSA compounds having ether linkages at one or more of the R₃, R₇, and R₁₂ positions;

FIG. 12C illustrate example CSA compounds having an amide linkage within the R₁₈ group and ether or urethane linkages at the R₃, R₇, and R₁₂ positions; and

FIG. 12D illustrate example CSA compounds having a glyceride linkage within the R₁₈ group and ester linkages at the R₃, R₇, and R₁₂ positions.

### DETAILED DESCRIPTION

### I. Introduction

Disclosed herein are compositions and methods for treating epidermolysis bullosa (EB) in a subject in need thereof by topically administering a treatment composition comprising one or more cationic steroidal antimicrobial (CSA) compounds and a carrier (“CSA treatment composition”). The treatment compositions can be applied to tissue with wounds or lesions in a patient suffering from EB or that may be at risk of developing EB wounds or lesions, including skin and mucosal tissue. Example forms of CSA treatment compositions include liquids, sprays, and gels, including spray gels, that can be applied topically to therapeutically or prophylactically treat EB wounds or lesions. The CSA treatment composition has been found to initiate wound healing, which is different than accelerating wound healing. That is because the body does not have a mechanism for healing EB wounds on its own.

Cationic steroidal anti-microbial (CSA) compounds, also referred to as “CSA compounds”, “CSAs”, “CSA molecules”, or “ceragenin” compounds, are synthetically produced, small molecule chemical compounds that include a sterol backbone having various charged groups (e.g., amine and cationic groups) attached to the backbone. The sterol backbone can be used to orient amine or guanidine groups on a face or plane of the sterol backbone. CSAs are cationic and amphiphilic, based upon the functional groups attached to the backbone. They are facially amphiphilic with a hydrophobic face and a polycationic face.

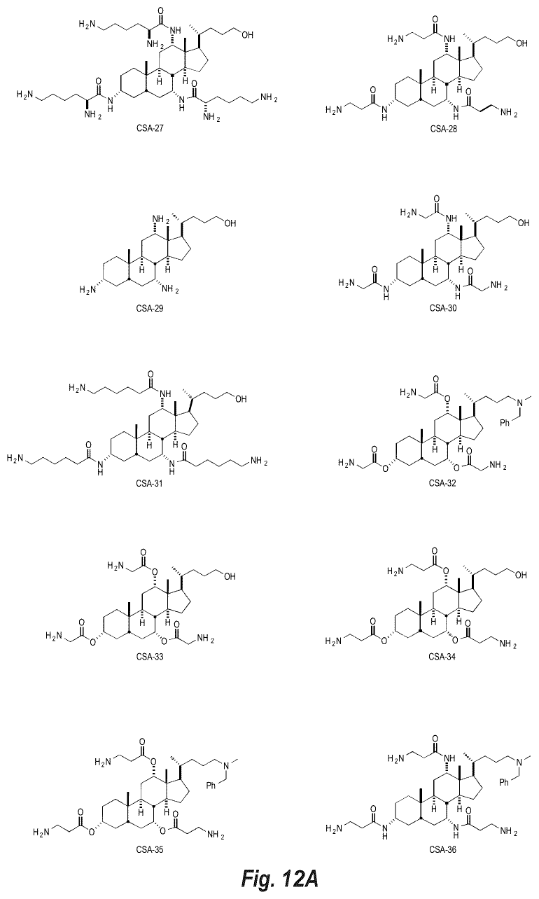

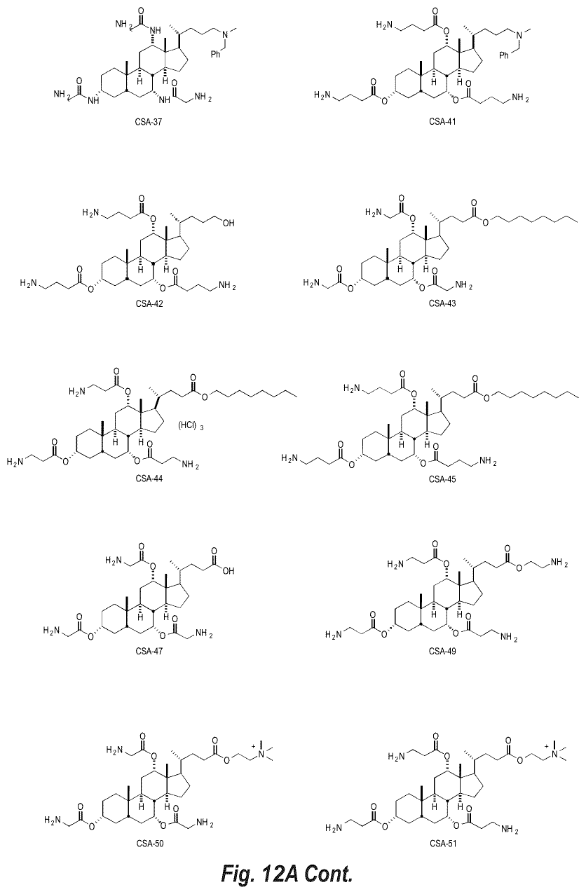

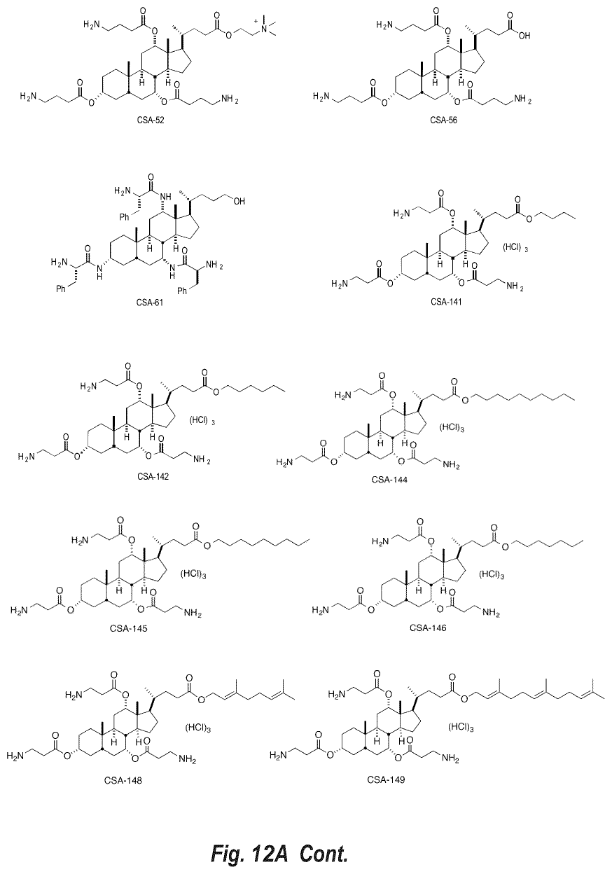

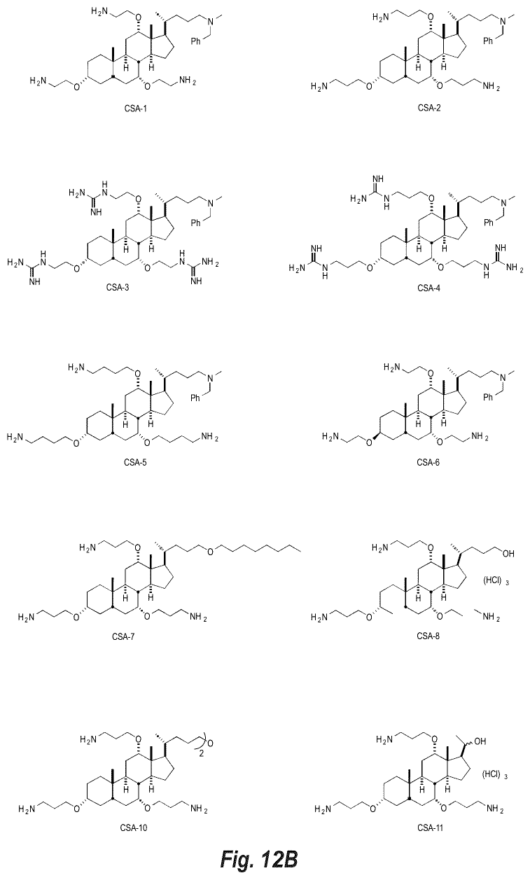

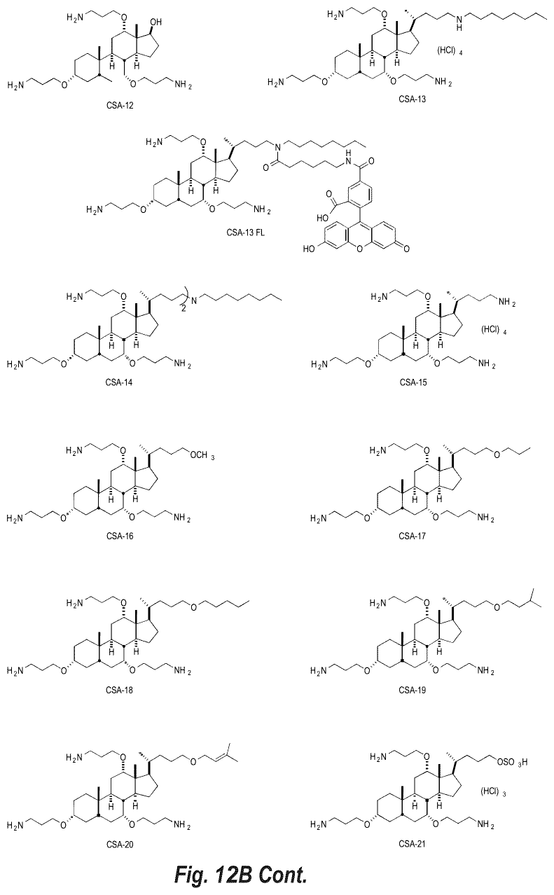

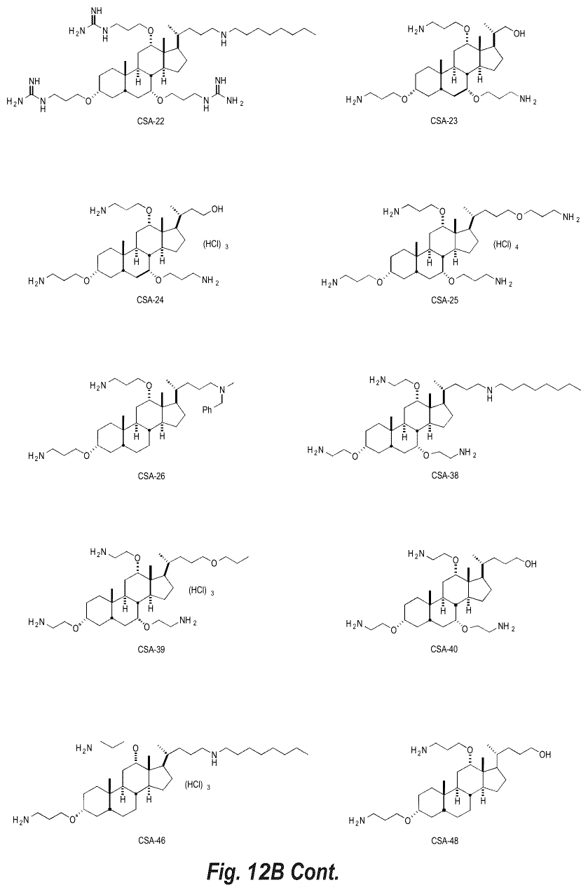

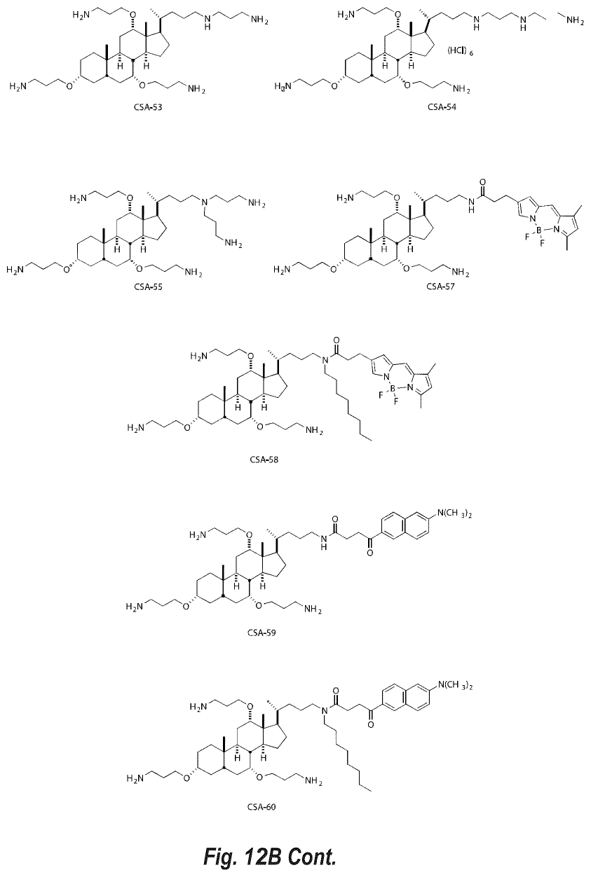

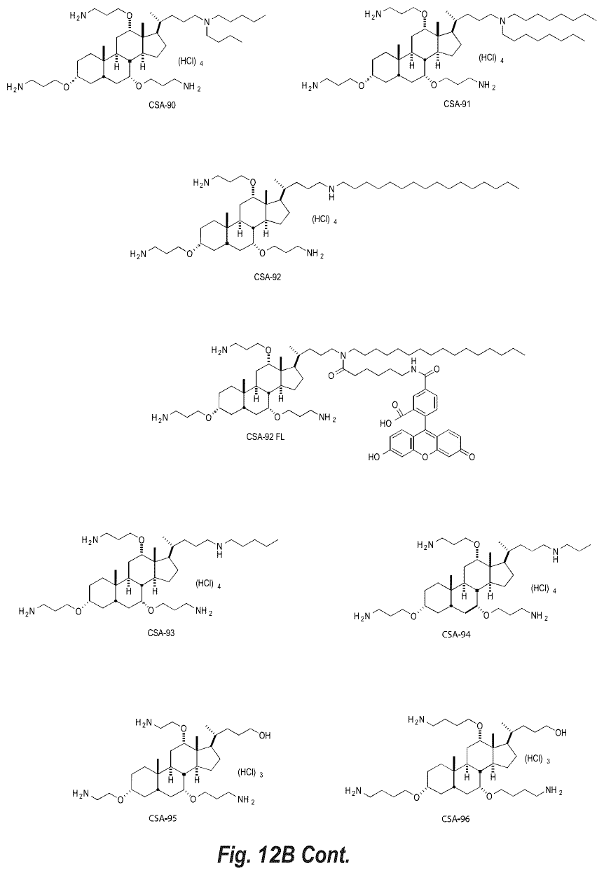

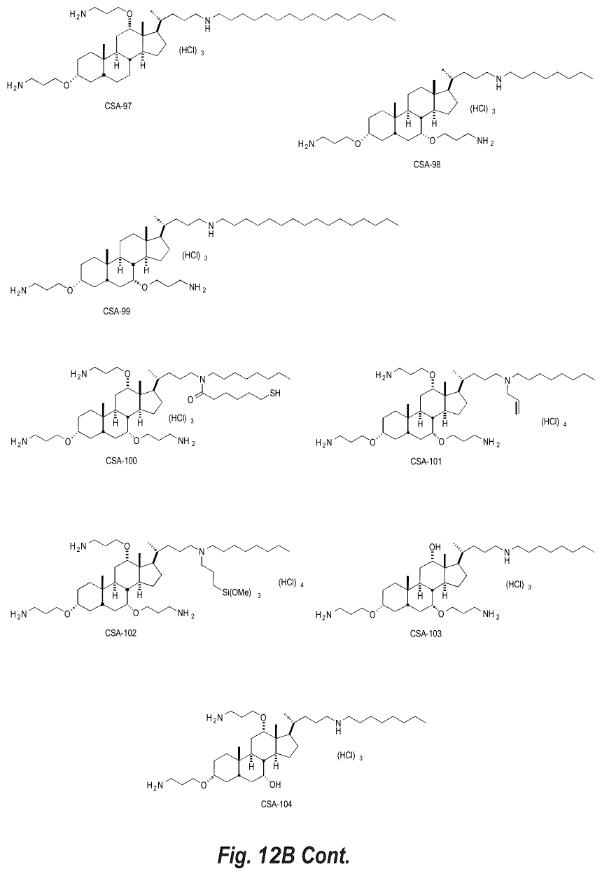

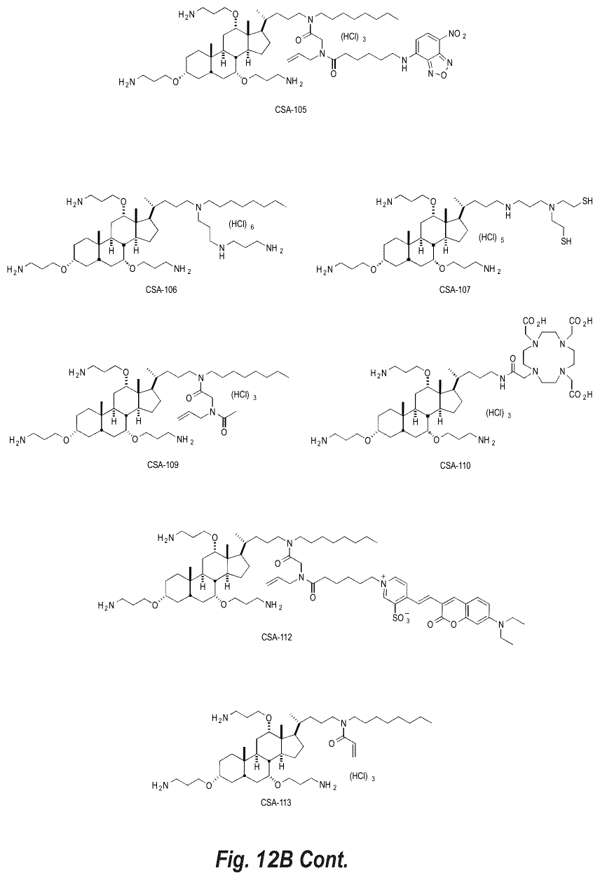

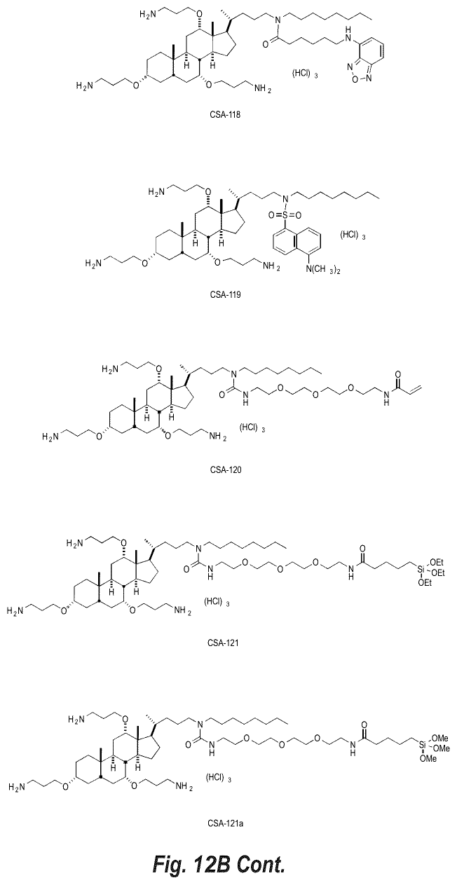

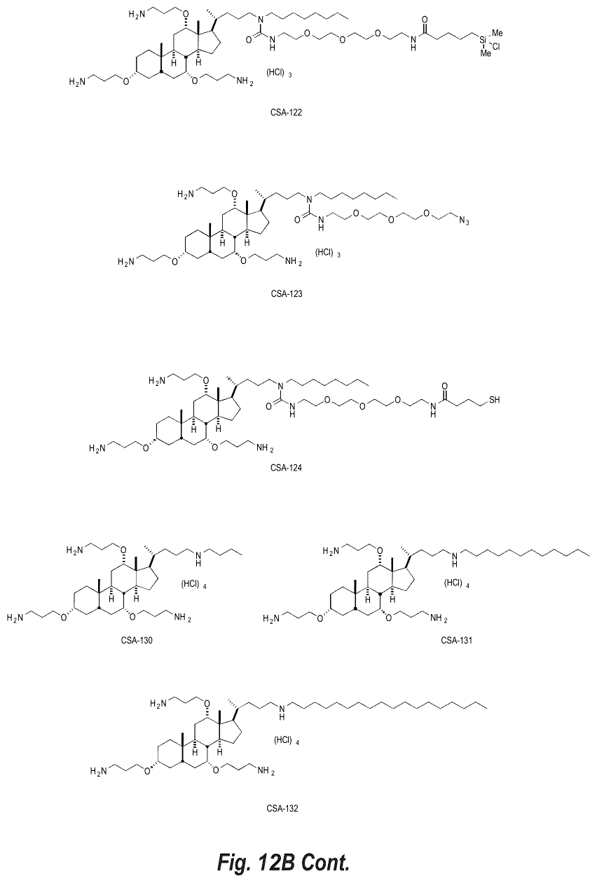

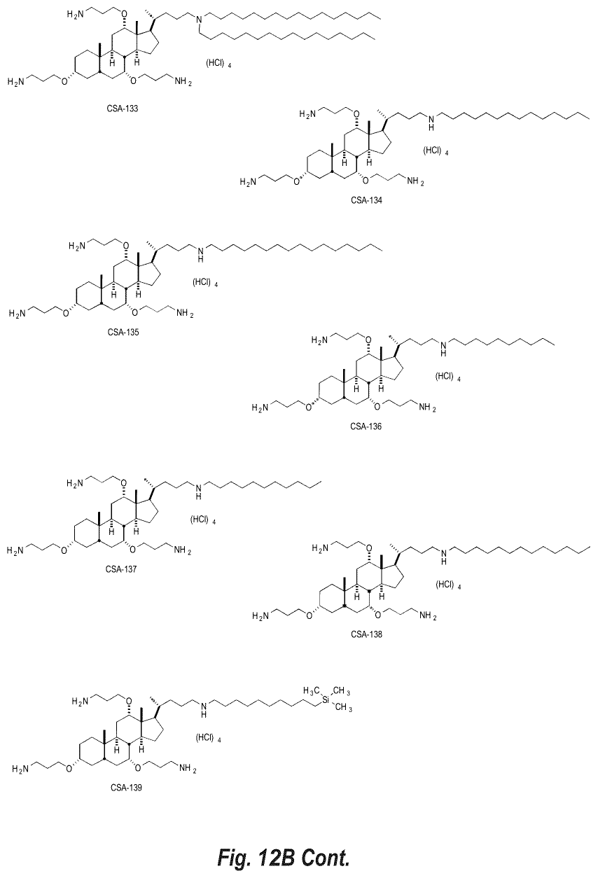

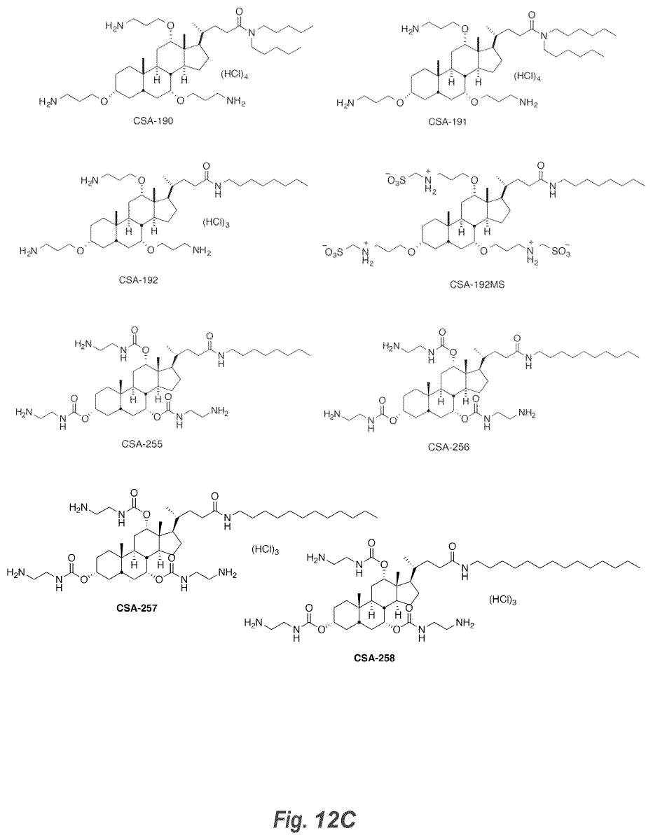

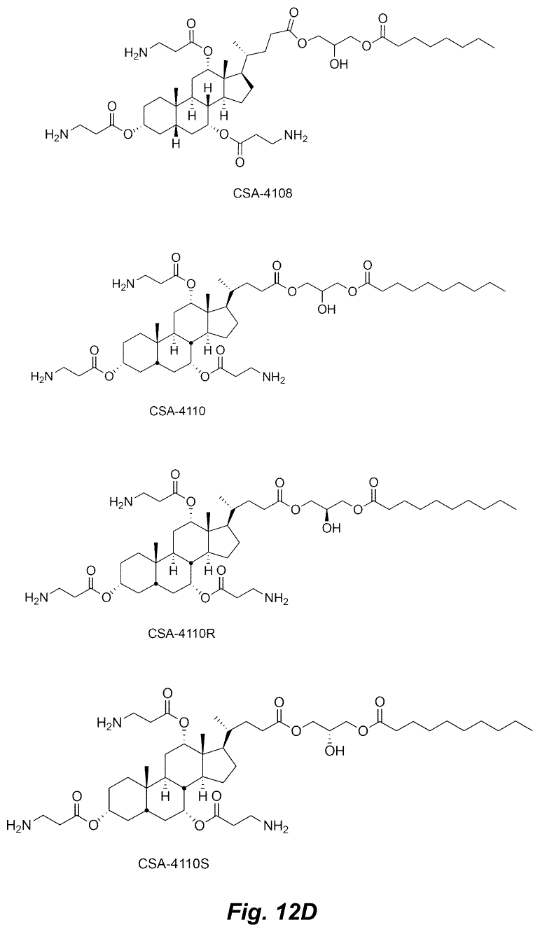

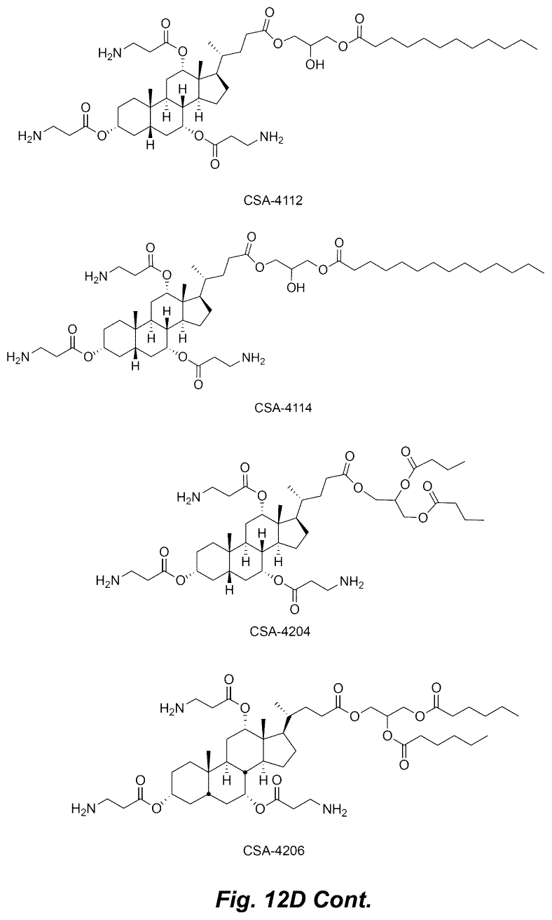

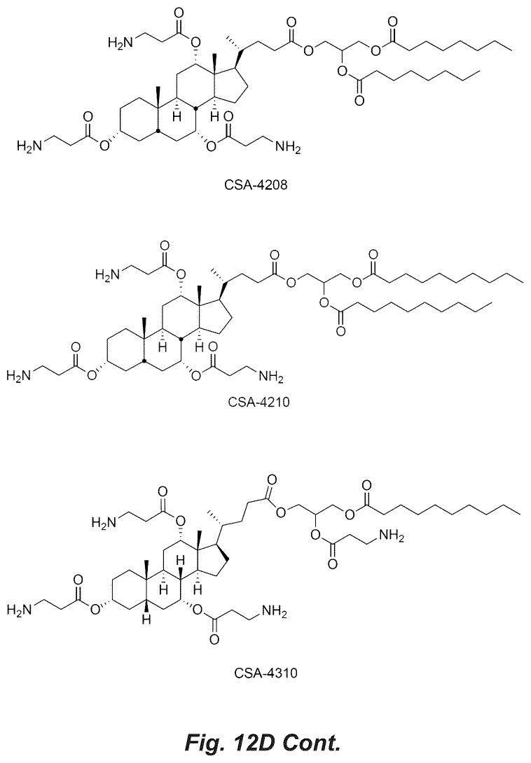

Examples of CSA compounds that can be included in the CSA treatment composition and used in the disclosed methods of treating EB are illustrated in FIGS. 12A-12D. A detailed description of CSA compounds that can be included in the CSA treatment composition and used in the disclosed methods of treating EB is set forth toward the end of this specification.

Typically, CSAs used herein can fall within two general categories: (1) CSAs having cationic groups linked to the sterol backbone with hydrolysable linkages and (2) CSAs having cationic groups linked to the sterol backbone with non-hydrolysable linkages. For example, one type of hydrolysable linkage is an ester linkage, and one type of non-hydrolysable linkage is an ether linkage. CSAs of the first type can be “inactivated” by hydrolysis of the linkages coupling the cationic groups to the sterol backbone, whereas CSAs of the second type are more resistant to degradation and inactivation.

A number of examples of CSA compounds that may be used in the embodiments described herein are illustrated in FIGS. 12A-12D. The general structures in free base (non-salt) form can be envisioned without the accompanying acid (when included), which forms no part of the compound unless otherwise specified. Non-limiting examples of CSAs with hydrolysable linkages are set forth in FIGS. 12A and 12D and include CSA-27, CSA-28, CSA-30, CSA-31, CSA-32, CSA-33, CSA-34, CSA-35, CSA-36, CSA-37, CSA-41, CSA-42, CSA-43, CSA-44, CSA-45, CSA-47, CSA-49, CSA-50, CSA-51, CSA-52, CSA-56, CSA-61, CSA-141, CSA-142, CSA-144, CSA-145, CSA-146, CSA-148, CSA-148, CSA-4108, CSA-4110, CSA-4110R, CSA-4110S, CSA-4112, CSA-4114, CSA-4204, CSA-4206, CSA-4208, CSA-4210, and CSA-4310. These CSA compounds in particular are very similar in structure and functionality such that test data generated using treatment compositions that contain CSA-44 is an accurate and reliable proxy for treatment compositions containing other CSA compounds of similar structure and functionality (e.g., that have the same stereochemistry as CSA-44 and amine functional groups at some or all of the R₃, R₇ and R₁₂ positions).

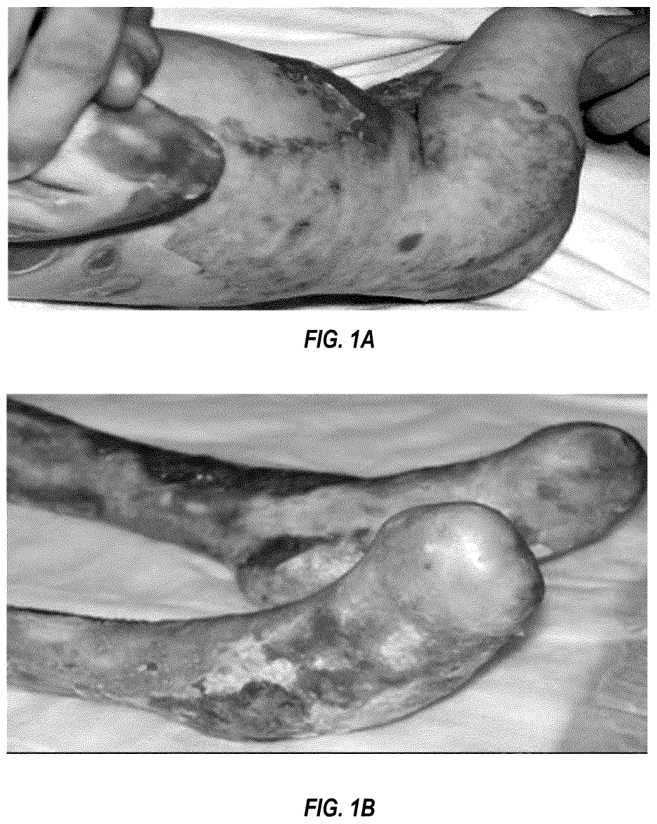

Non-limiting examples of CSAs with non-hydrolysable linkages are set forth in FIG. 12B and include CSA-1, CSA-2, CSA-3, CSA-4, CSA-5, CSA-6, CSA-7, CSA-8, CSA-9, CSA-10, CSA-11, CSA-12, CSA-13, CSA-13FL, CSA-14, CSA-15, CSA-16, CSA-17, CSA-18, CSA-19, CSA-20, CSA-21, CSA-22, CSA-23, CSA-24, CSA-25, CSA-26, CSA-38, CSA-39, CSA-40, CSA-46, CSA-48, CSA-53, CSA-54, CSA-55, CSA-57, CSA-58, CSA-59, CSA-60, CSA-90, CSA-91, CSA-92, CSA-92FL, CSA-93, CSA-94, CSA-95, CSA-96, CSA-97, CSA-98, CSA-99, CSA-100, CSA-101, CSA-102, CSA-103, CSA-104, CSA-105, CSA-106, CSA-107, CSA-109, CSA-110, CSA-112, CSA-113, CSA-118, CSA-119, CSA-120, CSA-121, CSA-121a, CSA-122, CSA-123, CSA-124, CSA-130, CSA-131, CSA-132, CSA-133, CSA-134, CSA-135, CSA-136, CSA-137, CSA-138, and CSA-139. Non-limiting examples of CSAs with both hydrolysable and non-hydrolysable linkages are set forth in FIG. 1C and include CSA-190, CSA-191, CSA-192, CSA-192MS, CSA-255, CSA-256, CSA-257, and CSA-258. These CSA compounds also have the same stereochemistry and include amine functional groups at some or all of the R₃, R₇ and R₁₂ such that test data generated using treatment compositions that contain CSA-44 is an accurate and reliable proxy for treatment compositions containing any of these CSA compounds,

In presently preferred embodiments, compositions used to therapeutically or prophylactically treat EB include one or more CSA compounds such as CSA-43, CSA-44, CSA-45, CSA-131, CSA-142, CSA-144, CSA-145, CSA-146, CSA-148, CSA-255, CSA-4108, CSA-4110, CSA-4110R, CSA-4110S, CSA-4112, CSA-4114, CSA-4204, CSA-4206, CSA-4208, CSA-4210, and CSA-4310, structurally similar CSA compounds, or combinations thereof.

### II. Treatment of EB Patients with CSA Treatment Compositions

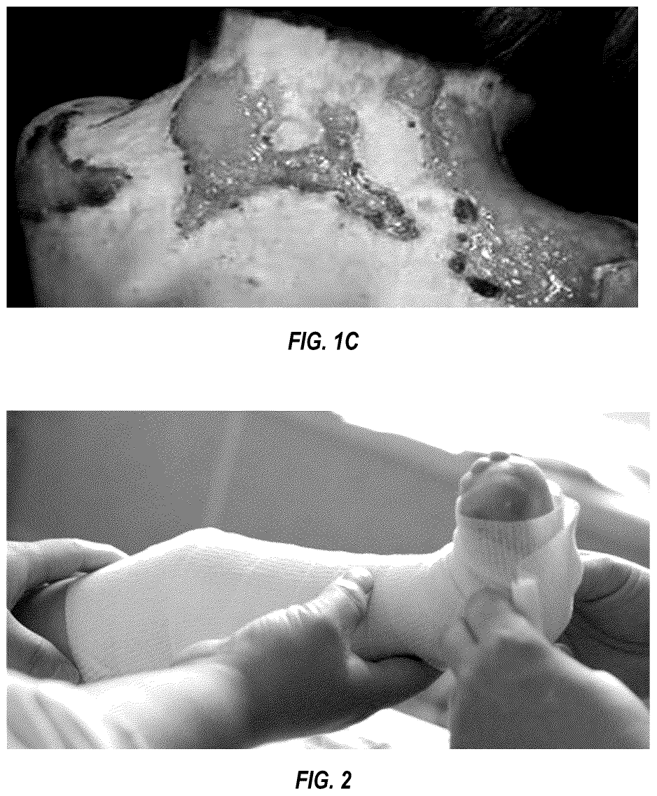

By way of background, FIGS. 1A-1C are photographs that illustrate badly damaged limbs and skin of infant patients suffering from epidermolysis bullosa (EB). Because there is no cure for EB, management of symptoms involves wound care, pain control, infection control, nutritional support, and prevention and treatment of complications. The main treatment option has been palliative treatment, mainly bandaging. FIG. 2 is a photograph that illustrates the bandaging of a limb of an infant suffering from EB. The foregoing figures show what a horrible disease EB is and the tremendous advancement in the art provided by the disclosed compositions and methods.

EB is caused by or the result of a mutation in at least one of 16 different genes. Some types are autosomal dominant while others are autosomal recessive. The underlying mechanism is a defect in attachment between or within the layers of the skin. Loss or diminished function of type VII collagen leads to weakness in the structural architecture of the dermal-epidermal junction (DEJ) and mucosal membranes. There are four main types of EB, which are epidermolysis bullosa simplex (EBS), dystrophic epidermolysis bullosa (DEB), junctional epidermolysis bullosa (JEB), and Kindler epidermolysis bullosa (KEB), formerly known as Kindler syndrome (KS).

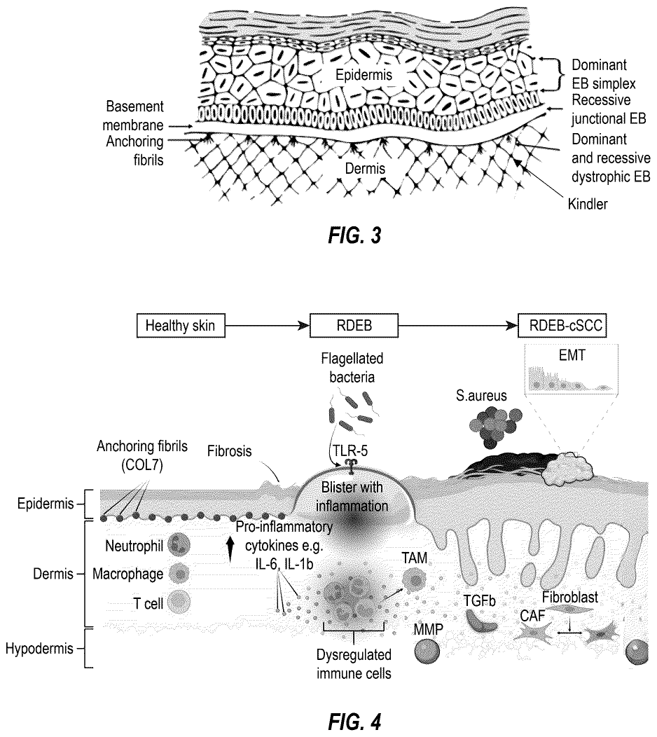

By way of further background, FIG. 3 is a diagram that schematically illustrates skin structure, including the different strata of skin, in a patient suffering from EB. The diagram shows the location of sites of primary blister formation, as well as the primary location of skin damage in the four types of EB. The epidermis is the primary location where skin damage is likely to occur in patients suffering from EB simplex. The junction between the epidermis and dermis is the primary location where skin damage is likely to occur in patients suffering from junctional EB. The upper dermis is the primary location where skin damage is likely to occur in patients suffering from dystrophic EB. The dermis in general is the primary location where skin damage is likely to occur in patients suffering from Kindler EB.

By way of further background, FIG. 4 is a diagram from Santucci, et al., “Unravelling drivers of cutaneous squamous cell carcinoma in recessive dystrophic epidermolysis bullosa,” Human Immunology 85 (2024) 110805. This diagram schematically illustrates and compares healthy skin, skin of a patient suffering from the recessive form of dystrophic EB (RDEB), and skin of a patient suffering from RDEB and with an aggressive form of squamous cell carcinoma (RDEB-cSCC). In the diagram, “CAY” is an abbreviation for cancer-associated fibroblast; “COL7” is an abbreviation for collagen VII; “EMT” is an abbreviation for epithelial to mesenchymal transition; “IL” is an abbreviation for interleukin; “MMP” is an abbreviation for matrix metalloproteinase(s); “RDEB-cSCC” is an abbreviation for recessive, dystrophic epidermolysis bullosa associated cutaneous squamous cell carcinoma(s); “RDEB” is an abbreviation for recessive dystrophic epidermolysis bullosa; “TAM” is an abbreviation for tumor associated macrophage(s); “TGF-β” is an abbreviation for transforming growth factor beta; “TLR” is an abbreviation for toll-like receptor(s).

FIG. 4 further illustrates skin layers corresponding to the epidermis, dermis, and hypodermis (or subcutaneous tissue) in both healthy skin (left third) and with EB blisters and lesions (middle and right thirds). In healthy skin (left third), anchoring fibrils (COL7) are located between the epidermis and dermis layers. Neutrophils, macrophages, and T cells are shown being present in the dermis of the healthy skin. Staphylococcus aureus (S. aureus) and flagellated bacteria are shown on or at the skin surface causing skin infections (middle and right thirds).

FIG. 4 further illustrates, in skin associated with RDEB (middle third), a blister with inflammation formed as a result of the interaction of flagellated bacteria and TLR-5. Fibrosis is shown next to the blister, indicating additional skin damage in the patient suffering from RDEB. Also present in the RDEB skin are pro-inflammatory cytokines, such as IL-6 and IL-1b, which are shown migrating toward the epidermis. Dysregulated immune cells are shown beneath the blister and cause, or are associated with, tumor associated macrophage(s) (TAM). Beneath the TAM are shown matrix metalloproteinase(s) (MMP).

FIG. 4 further illustrates, in skin associated with RDEB-cSCC (right third), epithelial to mesenchymal transition (EMT) formed adjacent to skin infected with S. aureus. Globules or legs of epidermis are illustrated extending into the dermis and a portion of the dermis extending into the hypodermis. Also illustrated are transforming growth factor beta (TGF-β), a cancer-associated fibroblast (CAF), and a fibroblast.

Recessive dystrophic EB (RDEB) is one of two forms of dystrophic EB (DEB). RDEB is caused by mutations in the gene COL7A, which encodes the extracellular matrix protein collagen VII (COL7). COL7 assembles into anchoring fibrils within the lamina densa of the basement membrane and works to adhere the epidermis and dermis together. The loss of COL7 triggers the detachment of the skin layers just beneath the lamina densa in the superficial dermis. In turn, this leads to blisters and chronic wounds, with extensive sequelae, such as tissue scarring and fibrosis, due to enduring inflammation, recurrent infections, as well as impaired wound healing, among other things. Ultimately, this greatly disrupts the normal architecture of skin. Due to the deep location of the defect in the skin, RDEB has a severe phenotype, and complications extend to extracutaneous systems and organs. Patients are also much more susceptible to infections that can develop into sepsis and death. Persistent itching and pain from inflamed, infected, non-healing wounds significantly diminish the quality of life for patients, and importantly, beyond the physical implications, there is also a humanistic burden to consider for both patients and their families.

Some of the most serious complications associated with RDEB are malignancies, especially an aggressive form of cutaneous squamous cell carcinoma (cSCC), which accounts for 97.6% of malignancies in this patient group. The first report of a squamous cell carcinoma (SCC) associated with EB came to light in 1913 and described the case of a 25-year-old female with a solitary lesion on the tongue, ultimately causing her death. Since then, a substantial body of evidence reinforcing the association between EB and SSC has been published. This is especially true in severe RDEB, where the median age of diagnosis is typically in the third decade of life, but can manifest as early as six years of age. Notably, for patients with severe generalized RDEB, the cumulative risk of a first cSCC reaches 90% and the cumulative risk of death reaches almost 80% by 55 years of age, making cSCC the leading cause of death in this patient group. RDEB-associated cSCCs (RDEB-cSCC) usually arise at sites prone to chronic wounding and scarring, with a predilection for extremities, particularly the legs and arms. The visual resemblance of this cancer to chronic ulceration complicates the clinical assessment and, since it frequently starts at the edge of a wound, only a segment of that wound might be malignant. Therefore, multiple biopsies may be needed for accurate diagnosis, but obtaining consent for this procedure can be challenging given patient hesitancy due to the associated pain. While this may certainly contribute to a delay in diagnosis and a resulting poorer prognosis, it is evident that there are other factors involved in the aggressive trait of this cancer.

For the treatment of RDEB-cSCC, surgical intervention, through excision of the lesion or amputation, remains the main management option, making up almost 90% of cases. Nevertheless, the median survival rate only reached between 60 and 72 months, and amputation entails significant adverse effects. Non-surgical management as a primary treatment modality of RDEB-cSCC has been tried and includes traditional chemotherapy; however, tumors tend to be poorly responsive, and risks may outweigh the benefits. Radiotherapy has also been used but, again, tends to have disappointing outcomes. Topical treatments such as imiquimod or 5-fluorouracil have shown some success in localized in situ cancer; however, the fragile skin of patients often makes such treatments impractical. Lastly, while electrochemotherapy and photodynamic therapy have demonstrated favorable results, their reliability varies.

Studies have shown that RDEB patients have a higher incidence of bacterial colonization, both in the upper respiratory tract and wounds, compared to healthcare workers and to non-EB patients with large wounds. It has been suggested that the loss of COL7, via cochlin from the lymphoid extracellular matrix, underlies a dysregulated innate immunity against bacteria. However, EB patient have high immunoglobulin G (IgG) levels, recognizing the most common bacterial antigen known to colonize EB wounds compared to healthy groups. This shows that immune systems in EB patients retain the ability to initiate a level of adaptive immune response against a pathogen, which is evidenced by relatively infrequent septic events in this patient group. Taken together, the evidence indicates that RDEB involves an intrinsic dysregulation of the systemic immune system and, most importantly, shifts the understanding of RDEB not just as a skin disease but also as a systemic inflammatory condition.

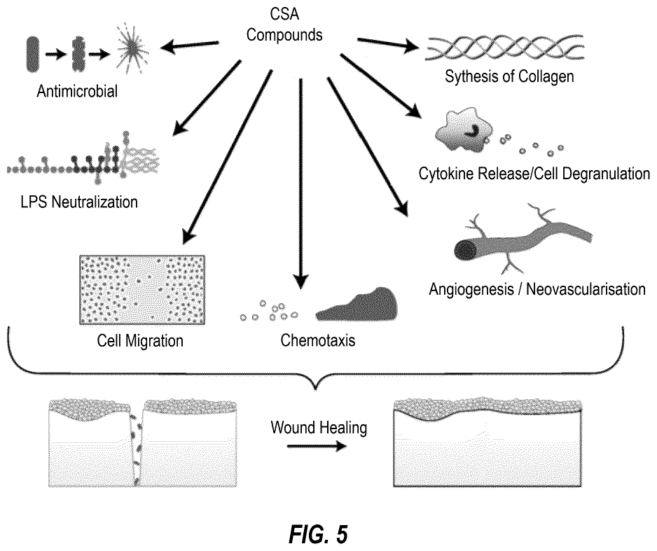

FIG. 5 is a diagram that illustrates proposed mechanisms of action by which CSA compounds in the CSA treatment compositions therapeutically or prophylactically treat skin and mucosal tissue (i.e., mucous membranes) of a patient suffering from EB, particularly as it relates to initiating healing of wounds and lesions, which otherwise does not occur in an EB patient, even when LL37 antimicrobial peptide is produced by the patient's body. In addition, CSA compounds are antimicrobial and therefore help reduce or eliminate microbes on the skin and mucous membranes that can cause infections and related damage. In addition, CSA compounds promote the synthesis of collagen, which is necessary for healing of skin and mucous membranes lesions and other damage. CSA compounds neutralize lipopolysaccharides (LPS), which can cause inflammation in skin and mucous membrane cells and contribute to atopic dermatitis. CSA compounds promote the release of cytokines, which help with cell degranulation. CSA compounds promote cell migration, chemotaxis, angiogenesis, and neovascularization to first initiate and then accelerate healing of EB lesions and wounds.

Thus, the three key activities promoted by treatment with CSA treatment compositions include: 1) initiation and acceleration of wound healing, which is promoted by CSA compounds triggering cell migration into wound beds and promoting neovascularization; 2) anti-inflammatory activity, such as by CSA compounds sequestering bacterial endotoxins, reducing the release of some endogenous cytokines, and up-regulating the release of IL-8; and 3) antimicrobial activity, such as bactericidal activity against Gram-positive and Gram-negative bacteria, including against drug-resistant organisms and established biofilms, fungicidal activity against pathogenic fungi, and antiviral activity against lipid-enveloped viruses.

It has been found by comparative testing that the beneficial activity of CSA compounds relative to treating patients suffering from EB greatly exceeds the activity of human LL-37, which does not provide significant healing of subjects with EB. CSA compounds have a broad impact on healing wounds, including initiating wound healing. CSA compounds signal through P2X7 and FPRL-1, generating the same activities observed with antimicrobial peptides: cell migration, neovascularization and acceleration of wound healing. CSA compounds also trigger host cell migration into wound beds. CSA compounds sequester endotoxins, reducing inflammation. CSA compounds stimulate angiogenesis. CSA compounds are broad spectrum antimicrobials with activity against Gram-positive and Gram-negative bacteria (including drug-resistant microbes) and fungi. CSA compounds are active against established biofilms (bacterial and fungal).

CSA treatment compositions used herein typically comprise a carrier and one or more CSA compounds dissolved and/or dispersed in the carrier. The carrier may be any suitable carrier in which the one or more CSA compounds can be mixed. Examples include water, aqueous solutions, alcohol and/or other organic solvents, emulsions, excipients, or combinations thereof. The CSA treatment compositions may be administered via any suitable route of administration including topically, orally, transdermally, parenterally, systemically, via inhalation, or via injection. Additional details regarding carriers, pharmaceutical compositions, and routes of administration are provided in another section below, though a few presently preferred embodiments are briefly described.

In some embodiments, the composition is formulated as a cream, liniment, salve, lotion, gel, liquid solution, spray, soap, or other such formulation readily administrable in a topical application. Topical administration can beneficially provide effective and long-lasting treatment for patients suffering from EB.

In some embodiments, the one or more CSA compounds are included in the treatment composition at a concentration by weight of about 100 ppm to about 1000 ppm, or about 150 ppm to about 900 ppm, or about 175 ppm to about 800 ppm, or about 200 ppm to about 750 ppm, or about 250 ppm to about 600 ppm, or about 300 ppm to about 500 ppm,

In some embodiments, the one or more CSA compounds are included at a concentration of about 10 μg/ml, about 25 μg/ml, about 50 μg/ml, about 75 μg/ml, about 100 μg/ml, about 125 μg/ml, about 150 μg/ml, about 175 μg/ml, about 200 μg/ml, about 300 μg/ml, about 350 μg/ml, about 400 μg/ml, about 450 μg/ml, about 500 μg/ml, about 550 μg/ml, about 600 μg/ml, about 650 μg/ml, about 700 μg/ml, about 750 μg/ml, about 800 μg/ml, about 850 μg/ml, about 900 μg/ml, about 950 μg/ml, or about 1000 μg/ml, or within a range defined by any two of the foregoing concentrations.

It will be understood that in the foregoing examples, the upper concentration endpoints do not necessarily represent a lack of effectiveness at CSA concentrations beyond the upper endpoints. Rather, the upper range endpoints define ranges for which effective activity may be achieved without the need for additional CSA compounds, thereby providing efficient use of CSA compounds given the associated formulation costs. In some implementations, such as where costs are less important than providing greater activity, the one or more CSA compounds may be included at concentrations higher than the foregoing ranges.

In some embodiments, the treatment composition is administered orally, such as by administering a pre-measured amount of a liquid treatment composition, which is swished around the patient's mouth (e.g., for about 10-60 seconds, or about 15-50 seconds, or about 20-40 seconds), which ensures the composition contacts all lesions in the mouth, and then swallowed to ensure the composition contacts lesions deep in the mouth and/or throat. In such cases, the treatment composition should formulated to be safe for oral ingestion.

The composition can be administered orally to the mouth and throat or topically to the skin. When administered orally, it can be by a device that dispenses a pre-measured quantity of liquid treatment composition as a bolus or a spray (e.g., more or multiple sprays), such as about 0.1 oz (3 mL) to about 1.5 oz (44.4 mL), or about 0.25 oz (7.4 mL) to about 1.25 oz (37 mL), or about 0.4 oz (11.8 mL) to about 1 oz (29.6 mL), or about 0.5 oz (14.8 mL) to about 0.8 oz (23.7 mL).

In other embodiments, the treatment composition can be applied topically to skin outside the mouth. In such cases, the quantity will depend on the surface area of skin to be treated.

Any CSA compound described herein, or any combination of such CSA compounds, may be utilized in a deactivation composition. In some circumstances, it may be preferable to administer one or more CSA compounds having hydrolysable linkages. Exemplary compounds include CSA-27, CSA-28, CSA-29, CSA-30, CSA-31, CSA-32, CSA-33, CSA-34, CSA-35, CSA-36, CSA-37, CSA-41, CSA-42, CSA-43, CSA-44, CSA-45, CSA-47, CSA-49, CSA-50, CSA-51, CSA-52, CSA-56, CSA-61, CSA-141, CSA-142, CSA-144, CSA-145, CSA-146, CSA-148, CSA-4108, CSA-4110, CSA-4110R, CSA-4110S, CSA-4112, CSA-4114, CSA-4204, CSA-4206, CSA-4208, CSA-4210, and CSA-4310, and in particular CSA-44, CSA-142, CSA-144, and CSA-148. These CSA compounds can be hydrolyzed or deactivated more rapidly than CSA compounds with non-hydrolysable linkages and may therefore carry less risk of remaining too long in active form within the system of the subject or on the skin or mucous membrane.

On the other hand, in certain situations it may be preferable to utilize one or more CSA compounds with non-hydrolysable linkages where longer term protection is desired and/or carries less risk. Exemplary compounds include CSA-1, CSA-13, CSA-26, CSA-38, CSA-40, CSA-46, CSA-48, CSA-53, CSA-55, CSA-57, CSA-60, CSA-90, CSA-91, CSA-92, CSA-107, CSA-109, CSA-110, CSA-112, CSA-113, CSA-118, CSA-124, CSA-130, CSA-131, CSA-139, CSA-190, CSA-191, CSA-192, and CSA-255. Such compounds can remain active for longer periods of time on the skin or mucous membrane and/or systemically if administered in that manner.

In some embodiments, the CSA treatment composition is an aqueous spray comprising an aqueous carrier and about 0.01%, 0.02%, about 0.03%, about 0.04%, about 0.05%, about 0.06%, about 0.07%, about 0.08%, about 0.09%, or about 0.1%, by weight of one or more CSA compounds. The CSA treatment composition can be locally delivered (i.e., sprayed using a spray bottle or liquid dispenser or applied as a cream or gel) to skin or mucous membrane (e.g., mouth, nose, throat, and/or anus) with lesions or wounds in a patient suffering from EB to provide therapeutic treatment thereof. The CSA treatment composition can also be applied to the entire body of a patient suffering from EB to provide both therapeutic and prophylactic treatment.

In some embodiments, a method of therapeutically or prophylactically treating a human patient suffering from EB comprises: (1) providing a CSA treatment composition that includes one or more CSA compounds and a carrier (e.g., aqueous and/or gel carrier); (2) administering the CSA treatment composition to soft tissue (e.g., skin and/or mucous membrane) of the human patient that has or is at risk of having EB wounds or lesions; and (3) the CSA treatment composition initiating and/or accelerating healing of soft tissue wounds caused by EB and/or preventing or mitigating formation of lesions or soft tissue wounds caused by EB in the human patient. It will be appreciated that the CSA treatment composition can accelerate healing in tissue where wound healing has already been initiated by administering a CSA treatment composition.

In some embodiments, a method of therapeutically treating a human patient suffering from EB comprises: (1) providing a CSA treatment composition that includes one or more cationic steroidal antimicrobial (CSA) compounds and a carrier (e.g., aqueous and/or gel carrier); (2) administering the CSA treatment composition to lesions or soft tissue wounds (e.g., in the skin and/or mucous membrane) caused by EB in the human patient; and (3) the CSA treatment composition initiating and/or accelerating healing of the lesions or soft tissue wounds caused by EB in the human patient.

In some embodiments, a method of prophylactically treating a human patient suffering from EB comprises: (1) providing a CSA treatment composition that includes one or more cationic steroidal antimicrobial (CSA) compounds and a carrier (e.g., aqueous and/or gel carrier); (2) administering the CSA treatment composition to soft tissue of the human patient suffering from EB that is at risk of developing lesions or soft tissue wounds caused by EB; and (3) the CSA treatment composition preventing or mitigating formation of lesions or soft tissue wounds caused by EB in the human patient.

The CSA treatment composition, when applied to skin and mucous membranes prophylactically, helps prevent further breakouts of blisters, lesions, or other skin or mucous membrane wounds, thereby treating a patient suffering from EB prophylactically, which is still a form of active therapeutic treatment since without such treatment, the patient would almost certainly experience future breakouts of soft tissue wounds and most likely suffer severe consequences resulting from such breakouts.

Treating EB with the CSA treatment compositions disclosed herein have been found to be effective in providing at least three key activities, which can act both therapeutically and prophylactically. The first mechanism of action is the initiation of wound healing by triggering cell migration into wound beds and promoting neovascularization. The second mechanism of action is anti-inflammatory by sequestering bacterial endotoxins, reducing the release of some cytokines, and up-regulating the release of IL-8. The third mechanism of action is providing antimicrobial activity, including: (1) providing bactericidal activity against Gram-positive and Gram-negative bacteria, including providing activity against drug-resistant organisms and established biofilms; (2) providing fungicidal activity against pathogenic fungi; and (3) inactivating lipid-enveloped viruses.

Moreover, even though the disclosed CSA treatment compositions were proposed and tested because it was believed they could mimic the activity of human cathelicidin LL-37 (a cationic peptide), the CSA treatment compositions unexpectedly and unpredictably exceeded the activity and efficacy of LL-37 in treating patients suffering from EB. The CSA treatment compositions have a broad impact on healing lesions and wounds, particularly initiation of healing of lesions and wounds that are otherwise non-healing (i.e., they are non-healing even though LL-37 is produced by and may be present in the body of patients suffering from EB, indicating that LL-37 appears to be ineffective in initiating wound healing in patients with EB).

The CSA treatment compositions have been found to signal through P2X7 and FPRL-1, generating activities observed with antimicrobial peptides, including cell migration, neovascularization, and acceleration of wound healing. The CSA treatment compositions also trigger host cell migration into wound beds of EB lesions and wounds, which is unique to the tested CSA compounds. The CSA treatment compositions sequester endotoxins, reducing inflammation. The CSA treatment compositions stimulate angiogenesis. The CSA treatment compositions are broad spectrum antimicrobials with activity against Gram-positive and Gram-negative bacteria (including drug-resistant microbes) and fungi. The CSA treatment compositions are active against established biofilms (bacterial and fungal). Thus, the CSA treatment compositions are effective in treating patients suffering from EB that human cathelicidin LL-37 and other endogenous antimicrobial peptides appear to be incapable of providing.

### III. Examples

### Example 1

A pilot clinical trial was performed using a CSA treatment composition comprising 0.05% by weight of CSA-44 (500 μg/ml of CSA-44, 500 ppm) dissolved in a carrier comprising water, 2% propylene glycol, and 0.01% lactic acid in saline. The CSA treatment composition used in the pilot clinical trial was in the form of an aqueous spray. The CSA treatment composition was locally administered (i.e., sprayed using a spray bottle) to skin lesions and wounds in a child suffering from EB. When placed in the mouth, the composition is advantageously swished around for prescribed time period (e.g., 10-60 second) to ensure that all mouth lesions are contacted with the composition and then swallowed to contact EB lesions deep in the mouth and/or throat of the patient.

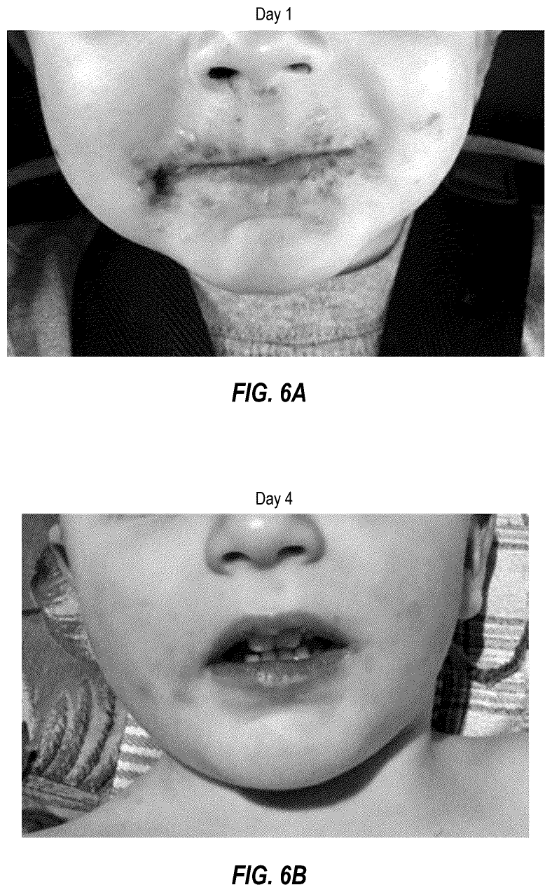

FIGS. 6A-6B are photographs of the mouth of a child suffering from EB Simplex at Day 1 and Day 4 of being treated with the CSA treatment composition comprising 0.05% by weight of CSA-44 in an aqueous carrier. The CSA treatment composition was applied by spraying onto the skin surrounding the child's mouth 1-2 times per day for 4 days. FIG. 6B shows that there was significant healing of EB wounds by Day 4. In fact, there was a dramatic reduction in both blisters and pain within 4 days.

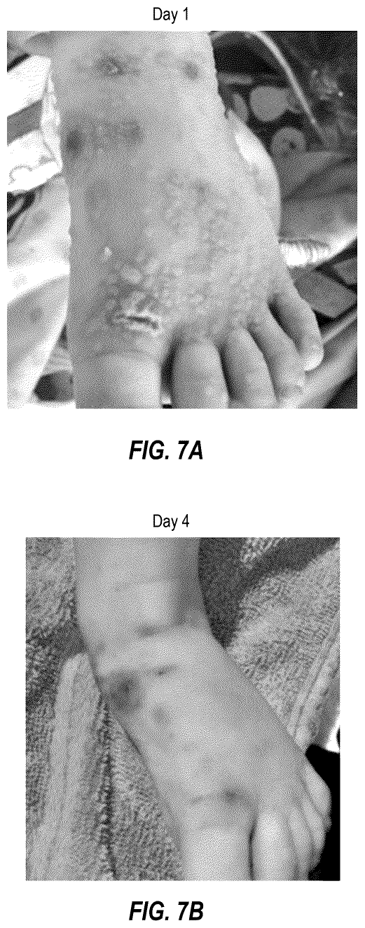

FIGS. 7A-6B are photographs of a foot of a child suffering from EB Simplex at Day 1 and Day 4 of being treated with the CSA treatment composition comprising 0.05% by weight of CSA-44 in an aqueous carrier. The CSA treatment composition was applied by spraying onto the skin of the child's foot 1-2 times per day for 4 days. FIG. 7B shows there was significant healing of EB wounds by Day 4. In fact, there was a dramatic reduction in both blisters and pain within 4 days.

### Example 2

This example involves a study entitled “Evaluation of Cav-1221 for Wound Healing in Epidermolysis Bullosa”, which was performed confidentially at the request of CSA BioTech, located in Spanish Fork, Utah and a licensee of the compositions and methods disclosed herein. The study tested if and how Cav-1221, a composition containing various concentrations of CSA-44, would affect metabolic activity and proliferation of RDEB SCC and RDEB Keratinocytes in vitro. The study utilized three different RDEB SCC cell lines, referred to as “RDEB SCC1”, “RDEB SCC62”, and “RDEB SCC63”, and one RDEB Keratinocyte cell line, referred to as “RDEB 223 Kc). Assays were performed using a concentration of 0.1-100 μg/ml of CSA-44.

A pilot clinical trial was performed using a CSA treatment composition comprising 0.05% by weight of CSA-44 (500 μg/ml, or 500 ppm, of CSA-44) dissolved in a carrier comprising water, 2% propylene glycol, and 0.01% lactic acid in saline. The CSA treatment composition used in the pilot clinical trial was in the form of an aqueous spray. (See FIGS. 6A-6B and 7A-7B for in vivo results in Example 1).

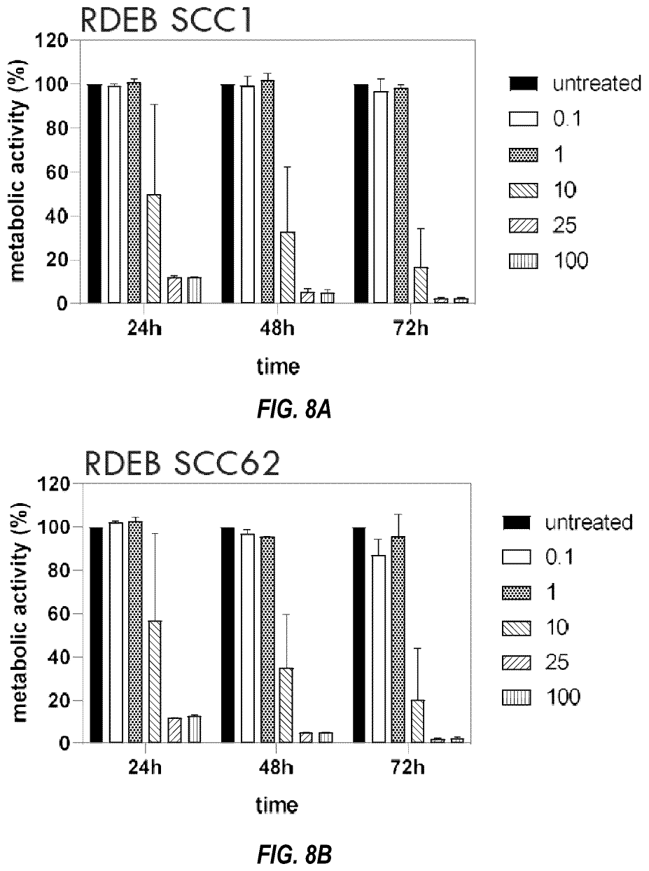

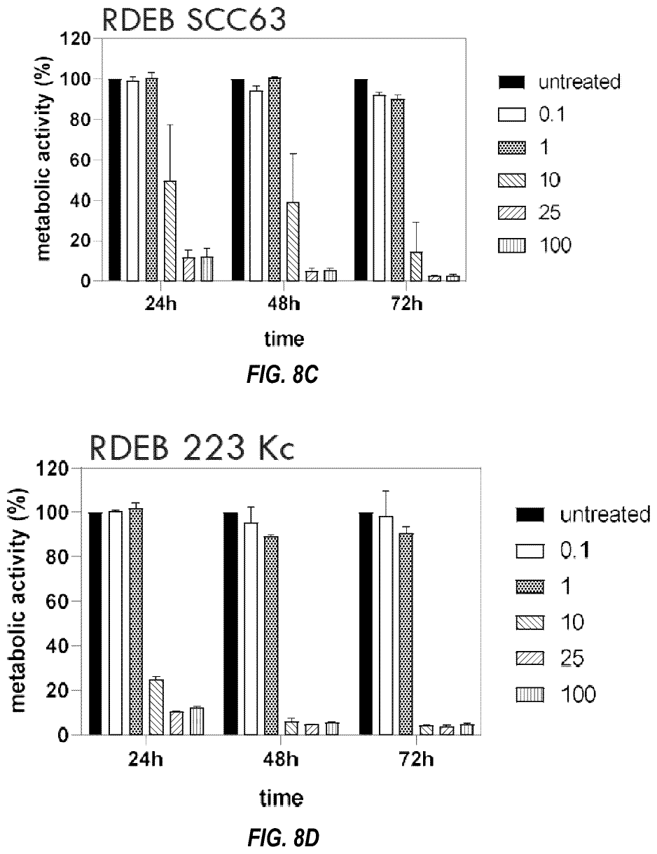

FIGS. 8A-8D are graphs that show the effect of Cav-1221 containing various concentrations (between 0.1-100 μg/ml, or 0.1-100 ppm) of CSA-44 on metabolic activity (%) of the different RDEB and RDEB SCC cell lines over time. Cav-1221 showed decreasing metabolic activity of all cell lines with increasing CSA-44 concentration, with the most dramatic decreases in metabolic activity at CSA-44 concentrations of 10 μg/ml (10 ppm) and 25 μg/ml (25 ppm), respectively.

FIG. 8A graphically illustrates the effect of CAV-1221, containing CSA-44 concentrations of 0.1 μg/ml (0.1 ppm), 1 μg/ml (1 ppm), 10 μg/ml (10 ppm), 25 μg/ml (25 ppm), and 100 μg/ml (100 ppm), respectively, on the metabolic activity (%) of RDEB SCC1 cells at time intervals of 0 hour, 24 hours, 48 hours, and 72 hours. Cav-1221 showed decreasing metabolic activity of RDEB SCC1 cells with increasing CSA-44 concentration, with the most dramatic decreases in metabolic activity at CSA-44 concentrations of 10 μg/ml (10 ppm) and 25 μg/ml (25 ppm), respectively, and greater.

FIG. 8B graphically illustrates the effect of CAV-1221, containing CSA-44 concentrations of 0.1 μg/ml, 1 μg/ml, 10 μg/ml, 25 μg/ml, and 100 μg/ml, respectively, on the metabolic activity (%) of RDEB SCC62 cells at time intervals of 0 hour, 24 hours, 48 hours, and 72 hours. Cav-1221 showed decreasing metabolic activity of RDEB SCC62 cells with increasing CSA-44 concentration, with the most dramatic decreases in metabolic activity at CSA-44 concentrations of 10 μg/ml (10 ppm) and 25 μg/ml (25 ppm), respectively, and greater.

FIG. 8C graphically illustrates the effect of CAV-1221, containing CSA-44 concentrations of 0.1 μg/ml, 1 μg/ml, 10 μg/ml, 25 μg/ml, and 100 μg/ml, respectively, on the metabolic activity (%) of RDEB SCC63 cells at time intervals of 0 hour, 24 hours, 48 hours, and 72 hours. Cav-1221 showed decreasing metabolic activity of RDEB SCC63 cells with increasing CSA-44 concentration, with the most dramatic decreases in metabolic activity at CSA-44 concentrations of 10 μg/ml (10 ppm) and 25 μg/ml (25 ppm), respectively, and greater.

FIG. 8D graphically illustrates the effect of CAV-1221, containing CSA-44 concentrations of 0.1 μg/ml, 1 μg/ml, 10 μg/ml, 25 μg/ml, and 100 μg/ml, respectively, on the metabolic activity (%) of RDEB 223 Kc cells at time intervals of 0 hour, 24 hours, 48 hours, and 72 hours. Cav-1221 showed decreasing metabolic activity of RDEB 223 Kc cells with increasing CSA-44 concentration, with the most dramatic decrease in metabolic activity at a CSA-44 concentration of 10 μg/ml (10 ppm) and greater.

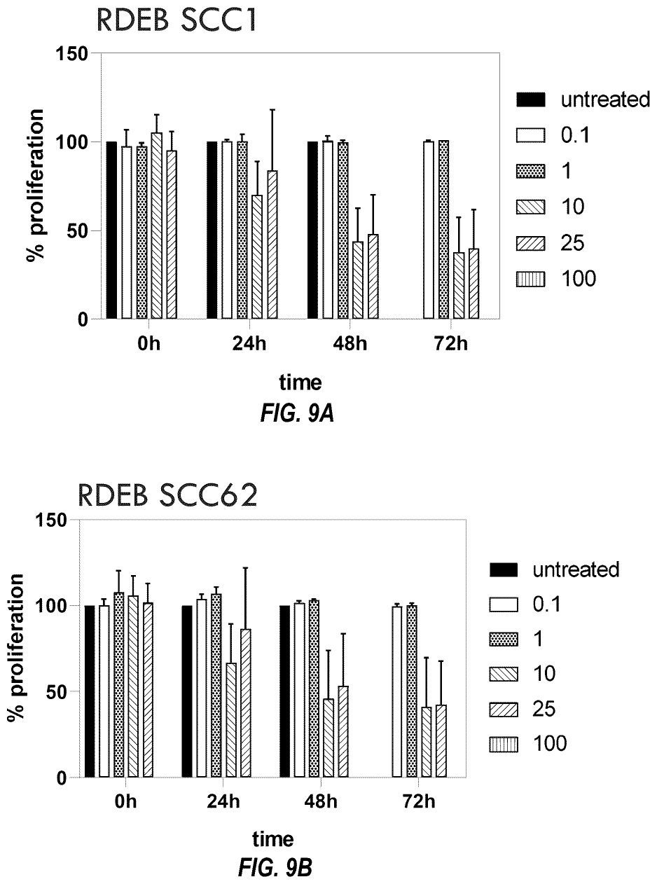

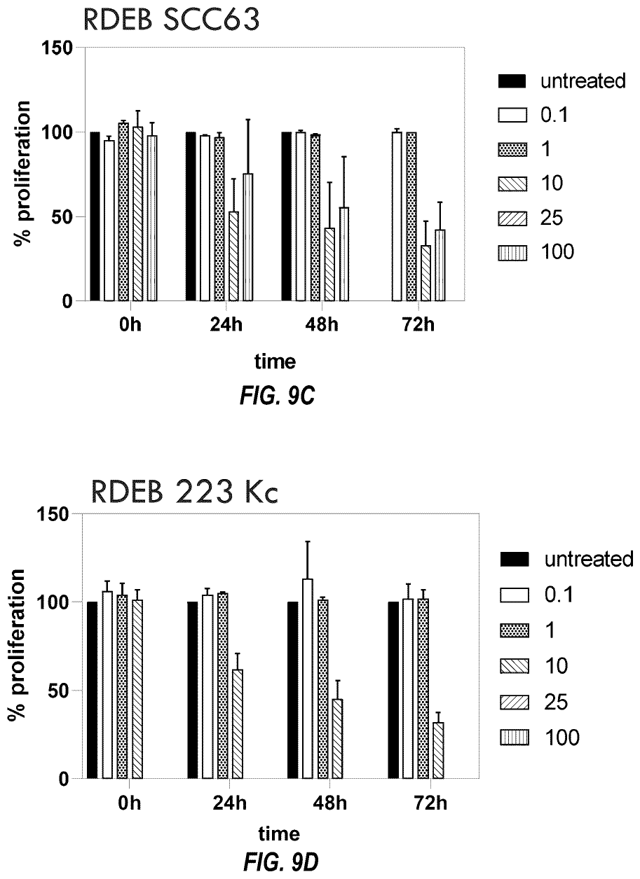

FIGS. 9A-9D are graphs that show the effect of Cav-1221 containing various concentrations (between 0.1-100 μg/ml) (0.1-100 ppm) of CSA-44 on % proliferation of the different RDEB and RDEB SCC cell lines over time. Cav-1221 showed decreasing proliferation of all cell lines with increasing CSA-44 concentration, with the most dramatic decrease in cell proliferation at CSA-44 concentrations of 10 μg/ml (10 ppm) and 100 μg/ml (100 ppm), respectively, and an anomalous decrease in cell proliferation at a CSA-44 concentration of 25 μg/ml (25 ppm) that was slightly less than the decrease in cell proliferation at a CSA-44 concentration of 10 μg/ml. There was no measurable proliferation of cells for all cell lines at a CSA-44 concentration of 100 μg/ml at all time intervals.

More particularly, FIG. 9A graphically illustrates the effect of CAV-1221, containing CSA-44 concentrations of 0.1 μg/ml, 1 μg/ml, 10 μg/ml, 25 μg/ml, and 100 μg/ml, respectively, on the % proliferation of RDEB SCC1 cells at time intervals of 0 hour, 24 hours, 48 hours, and 72 hours. Cav-1221 showed decreasing proliferation of RDEB SCC1 cells with increasing CSA-44 concentration, with the most dramatic decrease in cell proliferation at CSA-44 concentrations of 10 μg/ml and 100 μg/ml, respectively, and an anomalous decrease in cell proliferation at a CSA-44 concentration of 25 μg/ml that was slightly less than the decrease in cell proliferation at a CSA-44 concentration of 10 μg/ml. There was no measurable proliferation of RDEB SCC1 cells at a CSA-44 concentration of 100 μg/ml at all time intervals.

FIG. 9B graphically illustrates the effect of CAV-1221, containing CSA-44 concentrations of 0.1 μg/ml, 1 μg/ml, 10 μg/ml, 25 μg/ml, and 100 μg/ml, respectively, on the % proliferation of RDEB SCC62 cells at time intervals of 0 hour, 24 hours, 48 hours, and 72 hours. Cav-1221 showed decreasing proliferation of RDEB SCC62 cells with increasing CSA-44 concentration, with the most dramatic decrease in cell proliferation at CSA-44 concentrations of 10 μg/ml and 100 μg/ml, respectively, and an anomalous decrease in cell proliferation at a CSA-44 concentration of 25 μg/ml that was slightly less than the decrease in cell proliferation at a CSA-44 concentration of 10 μg/ml. There was no measurable proliferation of RDEB SCC62 cells at a CSA-44 concentration of 100 μg/ml at all time intervals.

FIG. 9C graphically illustrates the effect of CAV-1221, containing CSA-44 concentrations of 0.1 μg/ml, 1 μg/ml, 10 μg/ml, 25 μg/ml, and 100 μg/ml, respectively, on the % proliferation of RDEB SCC63 cells at time intervals of 0 hour, 24 hours, 48 hours, and 72 hours. Cav-1221 showed decreasing proliferation of RDEB SCC63 cells with increasing CSA-44 concentration, with the most dramatic decrease in cell proliferation at CSA-44 concentrations of 10 μg/ml and 100 μg/ml, respectively, and an anomalous decrease in cell proliferation at a CSA-44 concentration of 25 μg/ml that was slightly less than the decrease in cell proliferation at a CSA-44 concentration of 10 μg/ml. There was no measurable proliferation of RDEB SCC63 cells at a CSA-44 concentration of 100 μg/ml at all time intervals.

FIG. 9D graphically illustrates the effect of CAV-1221, containing CSA-44 concentrations of 0.1 μg/ml, 1 μg/ml, 10 μg/ml, 25 μg/ml, and 100 μg/ml, respectively, on the % proliferation of RDEB 223 Kc cells at time intervals of 0 hour, 24 hours, 48 hours, and 72 hours. Cav-1221 showed decreasing proliferation of RDEB 223 Kc cells with increasing CSA-44 concentration, with the most dramatic decreases in proliferation at CSA-44 concentrations of 10 μg/ml and 25 μg/ml, respectively. There was no measurable proliferation of RDEB 223 Kc cells at CSA-44 concentrations of 25 μg/ml and 100 μg/ml.

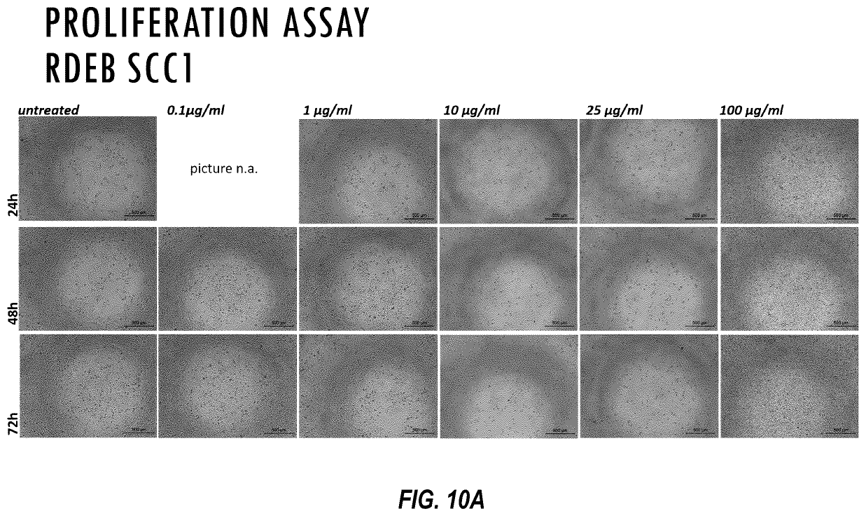

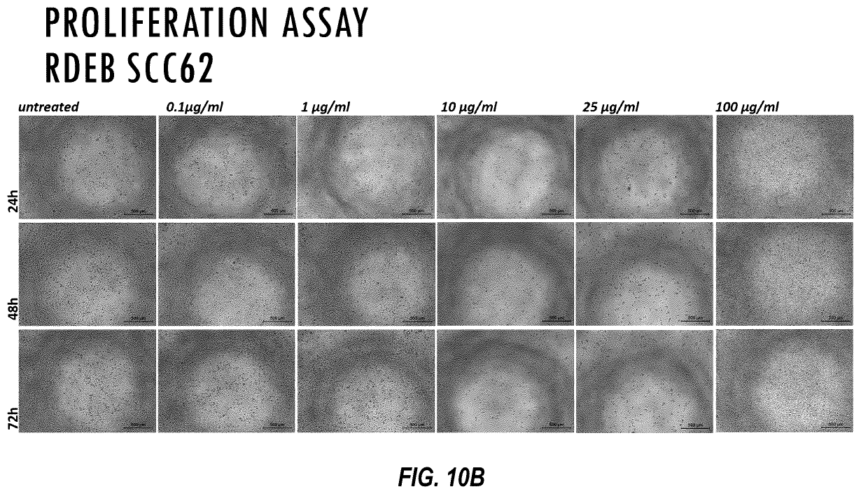

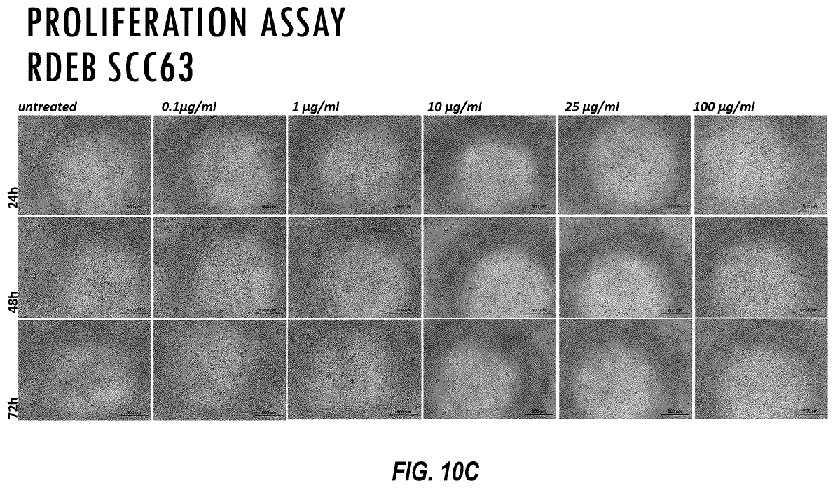

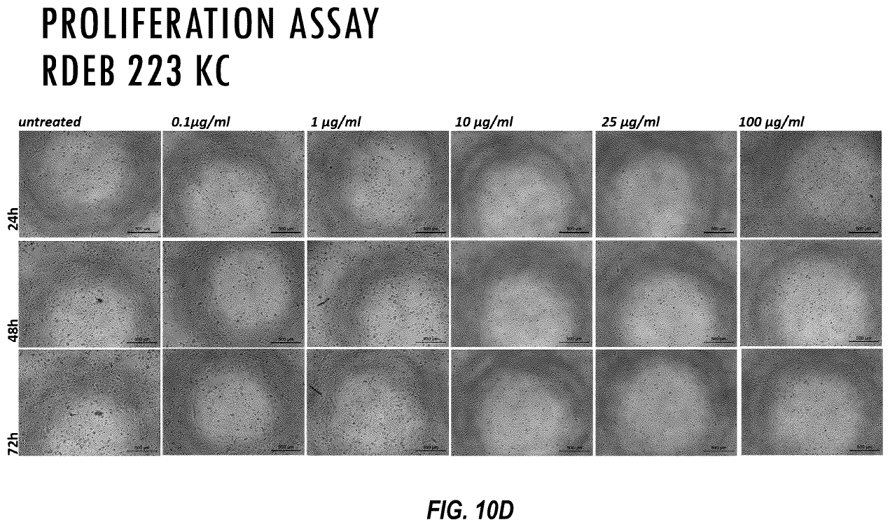

FIGS. 10A-10D are photographs that depict proliferation assays for the different cell lines at different CSA-44 concentrations and at the different time intervals, which were used in obtaining the data shown in the graphs of FIGS. 9A-9D.

FIG. 10A shows photographs that depict the proliferation assay for RDEB SCC1 at different CSA-44 concentrations and at the different time periods, which were used in obtaining the data shown in the graph of FIG. 9A.

FIG. 10B shows photographs that depict the proliferation assay for RDEB SCC62 at different CSA-44 concentrations and at the different time periods, which were used in obtaining the data shown in the graph of FIG. 9B.

FIG. 10C shows photographs that depict the proliferation assay for RDEB SCC63 at different CSA-44 concentrations and at the different time periods, which were used in obtaining the data shown in the graph of FIG. 9C.

FIG. 10D shows photographs that depict the proliferation assay for RDEB 223 Kc at different CSA-44 concentrations and at the different time periods, which were used in obtaining the data shown in the graph of FIG. 9D.

### Example 3

Room temperature stable topical aqueous and cream formulations containing CSA-44 for treatment of skin and mucous membrane conditions including blisters and other damage were proposed and prepared. The aqueous and cream formulations are likely to be effective in the treatment of EB by topical application, both therapeutically and prophylactically, on skin and mucous membranes. The aqueous and cream formulations are expected to initiate and promote healing of blisters and other wounds associated with EB. They are also expected to prevent or minimize breakouts when applied to the skin and/or mucous membranes (e.g., mouth, nose and/or anus) of an EB patient periodically, such as daily, every 2-3 days, every 4-5 days, or weekly.

CSA-44 is a synthetically produced small molecule consisting of a sterol backbone (cholic acid) with alanine and octanol attached to it. It is a hydrophilic molecule with a molecular weight of 543.41 Da and a theoretical Log P of 6.3. It is freely soluble in water (up to 5% w/v) and polar organic solvents. It is very hygroscopic and stability is pH dependent. The primary degradation pathway of CSA-44 is the loss of beta-alanine (ester hydrolysis) and octanol to form cholic acid.

CSA-44 has a net positive charge that is electrostatically attracted to the negatively charged cell membranes of certain viruses, fungi and bacteria. The high binding affinity for such cell membranes (including Lipid A) allow the CSA-44 to rapidly disrupt the target membranes leading to rapid cell death. CSA-44 can also form part of the body's innate immune system which helps it avoid many of the difficulties associated with its use as a therapeutic.

Current formulations include an aqueous formulation that contains 0.050% w/w CSA-44 (or other CSA compounds with hydrolysable linkages), 10 w/w propylene glycol, and 10% w/w Pluronic F127. Lactic acid (0.01% w/w) is included for pH adjustment. This formulation is efficacious but retention time on skin and mucous membranes is short. This example discusses the development of a stable cream product containing 1% w/w active and that should provide prolonged retention time at the intended treatment site with enhanced efficacy. Below in Table 1 is the target product profile:

TABLE 1
TARGET PRODUCT PROFILE

| | |
|---|---|
| Active Pharmaceutical | CSA-44 (or other CSA compound with |
| Ingredient (API) | hydrolysable linkages) |
| Indication | Topical skin and mucous membrane |
| | infections and blisters associated with EB |
| Dosage form | Topical cream formulation |
| Target concentration | 0.1% w/w (1000 ppm) |
| Target formulation pH | 4.5-5 |
| Dose/Dosing regimen | 1-20 mg dose |
| Formulation aesthetics/ | Cosmetically elegant formulation for |
| patient acceptability | better patient compliance: non-greasy, non- |
| | irritating, fast absorbing, topical formulation |
| | which does not have an odor and spreads |
| | easily on application |
| Packaging | Laminated tubes or pumps |
| Storage conditions | Ambient conditions |
| Shelf-life | ≥24 months |
| Efficacy End-points | Antimicrobial activity |
| Other | Non-toxic and non-irritating excipients |
| | only which are used in currently marketed |
| | products in the United States; FDA IIG listed |
| | limits for topical application |

Based on various tests and collected data, ten prototype compositions were prepared and named F1-F10. Their formulations are set forth in Table 2.

TABLE 2

| | | | | | | | | | | | | |
|---|---|---|---|---|---|---|---|---|---|---|---|---|
| | IID | | | | | | | | | | | |
| Excipients | limit | Function | F1 | F2 | F3 | F4 | F5 | F6 | F7 | F8 | F9 | F10 |

Oil Phase

| | | | | | | | | | | | | |
|---|---|---|---|---|---|---|---|---|---|---|---|---|
| Captex 300 | 70.00 | Oil base | | 31 | 32 | 30 | | 28 | | 25 | 26 | |
| (HLB: 5) | | | | | | | | | | | | |
| Light mineral | 39.92 | Oil base | 20 | | | | 25 | | 25 | | | 35 |
| oil (HLB: | | | | | | | | | | | | |
| 10.5) | | | | | | | | | | | | |
| Steareth-20 | 4.15 | Emulsifier | 4 | 4 | 4 | 4 | 4 | 4 | 4 | 4 | 4 | 4 |
| (HLB: 15.3) | | | | | | | | | | | | |
| Steareth-2 | 5.00 | Emulsifier | 3.5 | | | 3.5 | | | 3.5 | | | |
| (HLB: 4.9) | | | | | | | | | | | | |
| Glyceryl | 8.50 | Emulsifier | | | | | | 5 | | | 6 | |
| monostearate | | | | | | | | | | | | |
| (HLB: 3.8) | | | | | | | | | | | | |
| Span 60 | 6.00 | Emulsifier | | 6 | | | | | | 6 | | |
| (HLB: 4.7) | | | | | | | | | | | | |
| Span 80 | 7.00 | Emulsifier | | | 6 | | 6 | | | | | 6 |
| (HLB: 4.3) | | | | | | | | | | | | |
| Isopropyl | 15.00 | Penetration | 15 | 15 | 15 | | | 15 | 15 | 15 | | |
| Myristate | | Enhancer | | | | | | | | | | |
| Oleyl alcohol | 10.00 | Penetration | | | | 10 | 10 | | | 10 | 10 | 8 |
| | | Enhancer | | | | | | | | | | |
| Cetostearyl | 11.25 | Thickener | 7.5 | 7.5 | | 10 | | | 10 | 5 | | |
| alcohol | | | | | | | | | | | | |
| Cetyl alcohol | 12.00 | Thickener | | | | | 10 | 7 | | | | |
| Stearyl | 11.50 | Thickener | | | 8 | | | | | | 6 | 9 |
| alcohol | | | | | | | | | | | | |
| Cyclomethicone | 13.00 | Emollient | 5 | | 10 | | | 10 | 10 | | 10 | |
| Dimethicone | 2.30 | Emollient | | | | 2 | 2 | | | 2 | | 1.5 |
| copolyol | | | | | | | | | | | | |

API in Water phase

| | | | | | | | | | | | | |
|---|---|---|---|---|---|---|---|---|---|---|---|---|
| CSA-44 | NA | Active | 1 | 1 | 1 | 1 | 1 | 1 | 1 | 1 | 1 | 1 |
| Lactic Acid/ | NA | pH buffer; | 0.01 | 0.01 | 0.05 | 0.01 | 0.02 | 0.01 | 20 | 0.01 | 20 | 0.02 |
| 100 nM | | solvent | | | | | | | | | | |
| Acetate | | | | | | | | | | | | |
| Buffer (F7 | | | | | | | | | | | | |
| and F9) | | | | | | | | | | | | |
| Glycerin | 20.00 | Emollient | | 5 | | | | | | | | 5 |
| Transcutol ® | 15.00 | Permeation | 15 | 10 | | 15 | 12 | 10 | | | 15 | 7 |
| P | | enhancer | | | | | | | | | | |
| Water QS | | | 28.99 | 20.49 | 23.95 | 24.49 | 29.98 | 19.99 | 11.5 | 31.99 | 2 | 23.48 |
| Total | | | 100 | 100 | 100 | 100 | 100 | 100 | 100 | 100 | 100 | 100 |
| pH | | | 3.81 | 4.1 | 3.74 | 3.91 | 4.76 | 3.27 | 3.26 | 3.54 | 3.91 | 4.63 |

The 10 prototype formulations were placed on a short term stability study. Samples were pulled out at 2 weeks and 4 weeks to evaluate the physical and chemical stability (Table 3)

TABLE 3

| | 10 Formulation Prototypes | | |
|---|---|---|---|
| Storage | Time (weeks) | | |
| Condition | T₀ | 2 | 4 |
| 40° C. ± 2° C. / | C, P | C, P | C, P |
| 75% RH ± 5% RH | | | |
| 25° C. ± 2° C. / | C, P | C, P | C, P |
| 60% RH ± 5% RH | | | |

Short term stability results were detailed in the Table 4 and Table 5 below.

TABLE 4
Chemical Testing Results

| | | Assay Results (% LC) | | | | |
|---|---|---|---|---|---|---|
| | | | T = 2 Weeks | | T = 4 Weeks | |
| | Antibacterial | | 25° C./ | 40° C./ | 25° C./ | 40° C./ |
| Formulation | assay, % | T = 0 | 60% RH | 75% RH | 60% RH | 75% RH |
| F1 | 48 | 99.4 | 96.7 | 93.9 | 91.7 | 91.7 |
| F2 | 12 | 98.4 | 98.2 | 97.3 | 96.4 | 92.2 |
| F3 | 12 | 97.4 | 93.7 | 91.4 | 87.6 | 80.8 |
| F4 | 24 | 98.0 | 97.1 | 88.8* | * | * |
| F5 | 48 | 99.0 | 95.8 | 91.5* | * | * |
| F6 | 24 | 97.5 | 96.2 | 94.8 | 95.8 | 88.7 |
| F7 | 6 | 94.6 | 93.9 | 88.3 | 94.0 | 76.2 |
| F8 | 48 | 90.2 | 91.1 | 88.7 | 91.7 | 83.2 |
| F9 | 48 | 98.7 | 95.5 | 71.4* | * | * |
| F10 | 48 | 94.0 | 92.2 | 89.9 | 87.5 | 76.5 |

*F4, F5 and F9 were not tested for chemical stability due to phase separation.

TABLE 5
Physical testing results

| | | | T = 2 Weeks | | | | T = 4 Weeks | | | |
|---|---|---|---|---|---|---|---|---|---|---|
| | T = 0 | | 25° C./60% RH | | 40° C./75% RH | | 25° C./60% RH | | 40° C./75% RH | |
| | pH | Appearance | pH | Appearance | pH | Appearance | pH | Appearance | pH | Appearance |
| F1 | 3.81 | Normal, | 4.16 | Normal, | 4.13 | Normal, | 4.06 | Normal, | 3.94 | Normal, white, |
| | | white | | white | | white | | white | | |
| F2 | 4.1 | Normal, | 4.28 | Normal, | 4.24 | Normal, off | 4.47 | Normal, | 4.36 | Normal, |
| | | off white | | off white | | white | | off white | | yellowish, |
| F3 | 3.74 | Normal, | 3.79 | Normal, | 3.55 | Normal, | 3.84 | Normal, | 3.36 | Normal, |
| | | yellowish | | yellowish | | yellowish | | yellowish | | yellowish |
| F4 | 3.91 | Normal, | 4.10 | Normal, | 3.99 | Obvious | 4.36 | Normal, | 3.91 | Obvious phase |
| | | off white | | off white | | phase | | off white | | separation |
| | | | | | | separation | | phase | | observed |
| | | | | | | observed | | | | Obvious phase |
| F5 | 4.76 | Normal, | 4.86 | Phase | 4.63 | Phase | 4.79 | phase | 4.42 | separation, yellow |
| | | off white | | separation | | separation | | separation | | liquid at bottom |
| | | | | started | | started | | started | | |
| F6 | 3.27 | Viscous, | 3.81 | Viscous, | 3.62 | Viscous, off | 4.12 | Viscous, | 3.57 | Viscous, off white |
| | | off white | | off white | | white | | off white | | |
| F7 | 3.26 | Viscous, | 3.49 | Viscous, | 3.04 | Viscous, off | 3.57 | Viscous, | 2.99 | Viscous, off white |
| | | off white | | off white | | white | | off white | | |
| F8 | 3.54 | Normal, | 3.73 | Normal, | 3.51 | Normal, off | 3.72 | Normal, | 3.28 | Normal, off white |
| | | off white | | off white | | white | | off white | | |
| F9 | 3.91 | Normal, | 4.18 | Phase | 4.07 | Obvious | 4.27 | phase | 3.89 | Obvious phase |
| | | off white | | separation | | phase | | separation | | separation |
| | | | | started | | separation | | started | | observed |
| | | | | | | observed | | | | |
| F1 | 4.63 | Normal, | 4.79 | Normal, | 4.51 | Normal, off | 4.71 | Normal, | 4.50 | Normal, off white |
| | | off white | | off white | | white | | off white | | |

Based on the data, F1, F2 and F6 were identified as promising candidates with good physical and chemical stability. Phase separation was observed for F4, F5 and F9 formulations and therefore were not tested for drug content at 4 weeks. F3, F7, F8 and F10 have high levels of degradation at 4 weeks at accelerated conditions (40° C.) and therefore are not recommended.

According to functional efficacy data provided by the Sponsor, F1, F5, F8, F9 and F10 are the most promising candidates with 48% efficacy. Therefore, F1 became the best candidate for further evaluation and optimization. For the skin permeation study, it was recommended to evaluate F2 and F6 formulations along with F1 formulation. Even though F2 and F6 formulations have comparatively lower values for antimicrobial activity (12% and 24% respectively) after 1 hour incubation, it could be beneficial to have the API released more slowly from the formulations. In addition, F1, F2 and F6 consist of different levels of penetration enhancers which may render different permeation profiles.

The manufacturing procedure for making F1, F2, and F6 cream formulations included the following steps:
1) Weigh all the excipients listed in the oil phase category individually and mix in glass jar #1 (oil phase);
2) Weigh API, lactic acid, Transcutol® P, and Water (˜10%) in glass jar #2 (water phase A);
3) Weigh glycerin and the rest of water in glass jar #3 (water phase B);
4) Heat both oil phase and water phase B to ˜70° C.;
5) Add the water phase B to the oil phase while homogenization at 6 k rpm for 5 minutes using IKA homogenizer;
6) Allow the formulations to cool down while stirring at 100 rpm using an overhead IKA mixer;
7) Once the temperature of the formulations drops down to 40-45° C., add water phase A to the formulation while homogenize at 12 k rpm for 5-10 minutes; and
8) Cool down the cream formulation to room temperature while stirring at 100 rpm using an overhead mixer.

Thereafter, a skin permeation study was conducted. In this study model, dermatomed human cadaver skin was mounted onto vertical diffusion cells chambers and the tissue was maintained at typical temperature and humidity conditions that represent in vivo conditions. The test dosage form was applied to the outer surface of the tissue and drug absorption was measured by monitoring the rate of appearance of the active in the receiving medium bathing the inner surface of the tissue. The compositions of the test formulations are set forth in Table 6.

TABLE 6

| | | | | Current lead |
|---|---|---|---|---|
| Excipients | F1 | F2 | F6 | formulation |
| Captex ® 300 (HLB: 5) | — | 31.00 | 28.00 | |
| Light mineral oil (HLB: 10.5) | 20.00 | — | — | |
| Steareth-20 (HLB: 15.3) | 4.00 | 4.00 | 4.00 | |
| Steareth-2 (HLB: 4.9) | 3.50 | — | — | |
| Glyceryl monostearate (HLB: 3.8) | — | — | 5.00 | |
| Span 60 (HLB: 4.7) | — | 6.00 | — | |
| Isopropyl Myristate | 15.00 | 15.00 | 15.00 | |
| Cetostearyl alcohol | 7.50 | 7.50 | — | |
| Cetyl alcohol | — | — | 7.00 | |
| Cyclomethicone | 5.00 | — | 10.00 | |
| CSA-44 | 1.00 | 1.00 | 1.00 | 0.05 |
| Lactic Acid | 0.01 | 0.01 | 0.01 | 0.01 |
| Glycerin | — | 5.00 | — | |
| Transcutol ® P | 15.00 | 10.00 | 10.00 | |
| Propylene glycol | | | | 1.00 |
| Pluronic F127 | | | | 1.00 |
| Purified Water | 28.99 | 20.49 | 19.99 | 97.94 |

A pilot run was performed prior to the pivotal study with F1 to evaluate expected permeation profiles, drug levels in the dermis (site of action) and determine the critical sampling time points for the pivotal study. Specifics of the pilot study design are detailed in protocol P-CSA-003. After 24 hour application, the amounts of CSA-44 analyzed in the receptor chamber were all lower than the limit of detection (LOD, 50 ng/mL). Approximately 10% of dosed API can be detected in the skin tissue, and mostly concentrated in the epidermis.

A pivotal in vitro permeation study was performed to screen the permeation efficiency of the active from select formulations (F1, F2, and F76). Specifics of the pivotal study design are detailed in protocol P-CSA-004.

For all four test formulations, there were no API detected in the receptor media after 24 h application which is consistent with the pilot study. Within the skin tissue, the three cream formulations (F1, F2, and F6) have shown significantly higher amount of drug releases in both epidermis and dermis compared to that of the current clinical product. Most of the active was concentrated in the dermis for all treatment groups, and F1 formulation showed much higher drug exposure than F2 and F6. Overall, based on the result of stability, IVPT, and the functional efficacy study (provided by the sponsor), it was recommended to proceede with F1 formulation for further studies.

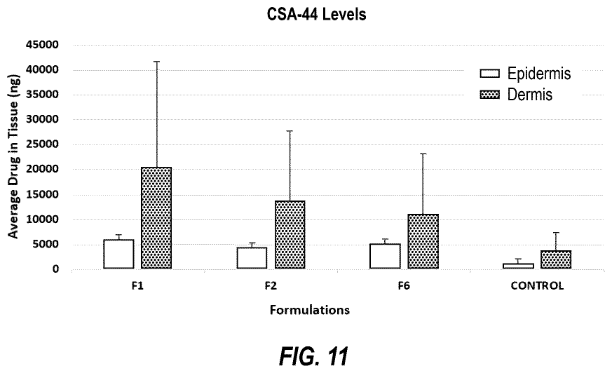

FIG. 11 is a graph that illustrates the amounts of CSA-44 in skin tissues for the F1, F2 and F6 formulations compared to a control.

### Example 4

Examples 1-3 are each modified by substituting CSA-44 with another CSA compound selected from: CSA-27, CSA-28, CSA-29, CSA-30, CSA-31, CSA-32, CSA-33, CSA-34, CSA-35, CSA-36, CSA-37, CSA-41, CSA-42, CSA-43, CSA-44, CSA-45, CSA-47, CSA-49, CSA-50, CSA-51, CSA-52, CSA-56, CSA-61, CSA-141, CSA-142, CSA-144, CSA-145, CSA-146, CSA-148, CSA 4108, CSA 4110, CSA-4110R, CSA-4110S, CSA-4112, CSA-4114, CSA-4204, CSA-4206, CSA-4208, CSA-4210, and CSA-4310, and in particular CSA-44, CSA-142, CSA-144, and CSA-148. The treatment compositions provide similar efficacy when used to treat EB as the compositions containing CSA-44. It will be appreciated that the skilled person can adjust the concentration the CSA compound to obtain the same or similar efficacy when treating EB as when using the compositions containing CSA-44.

### Example 5

Examples 1-3 are each modified by substituting CSA-44 with another CSA compound selected from: CSA-1, CSA-13, CSA-26, CSA-38, CSA-40, CSA-46, CSA-48, CSA-53, CSA-55, CSA-57, CSA-60, CSA-90, CSA-91, CSA-92, CSA-107, CSA-109, CSA-110, CSA-112, CSA-113, CSA-118, CSA-124, CSA-130, CSA-131, CSA-139, CSA-190, CSA-191, CSA-192, and CSA-255. The treatment compositions provide similar efficacy when used to treat EB as the compositions containing CSA-44. It will be appreciated that the skilled person can adjust the concentration the CSA compound to obtain the same or similar efficacy when treating EB as when using the compositions containing CSA-44.

### IV. Additional Details of CSA Compounds

Exemplary CSA compounds that can be used in the treatment compositions and treatment methods disclosed, and methods for their manufacture, are described in U.S. Pat. Nos. 6,350,738, 6,486,148, 6,767,904, 7,598,234, 7,754,705, 8,691,252, 8,975,310, 9,434,759, 9,527,883, 9,943,614, 10,155,788, 10,227,376, 10,370,403, 10,626,139, 11,286,276, 12,030,912 and 12,215,126; and U.S. Pat. Pub. Nos. 2016/0311850 and 2028/0073248, which are incorporated herein by reference. The skilled artisan will recognize the compounds within the generic formulae set forth herein and understand their preparation in view of the references cited herein and the Examples contained therein.

CSA compounds can have a structure of Formula I, Formula II, Formula III, and/or Formula IV. Formula III differs from Formula I and II by omitting R₁₅ and the ring carbon to which it is attached. Formula IV more particularly defines Formula III with respect to stereochemistry and R groups for all but R₃, R₇, R₁₂, and R₁₈.

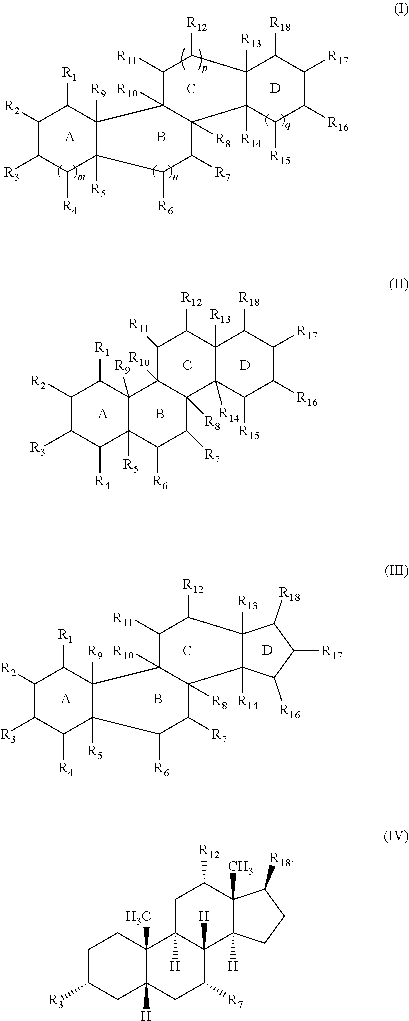

In some embodiments of Formulas I, II, III, and IV, at least two of R₃, R₇, and R₁₂ may independently include a cationic moiety (e.g., amino or guanidino groups) bonded to the steroid backbone structure via a hydrolysable or non-hydrolysable linkage. For the embodiments of the present disclosure, the linkage is preferably hydrolysable but stable under conditions of sterilization and storage, and hydrolysable under physiological conditions. Such cationic functional groups (e.g., amino or guanidino groups) may be separated from the backbone by at least one, two, three, four or more atoms.

A tail moiety may be attached to the sterol backbone at R₁₈, may have variable chain length or size, and may be charged, uncharged, polar, non-polar, hydrophobic, or amphipathic. The tail moiety may be used to select the hydrophobicity/hydrophilicity of the ceragenin compound. CSA compounds having different degrees of hydrophobicity/hydrophilicity may have different rates of uptake into different target microbes.

The “R” groups described herein, unless specified otherwise, may be substituted or unsubstituted.

With respect to CSA compounds of Formulas I, II, and III (and where not already specified with respect to Formula IV):
each of fused rings A, B, C, and D may be independently saturated, or may be fully or partially unsaturated, provided that at least two of A, B, C, and D is saturated, wherein rings A, B, C, and D form a ring system. Other ring systems can also be used, e.g., 5-member fused rings and/or compounds with backbones having a combination of 5- and 6-membered rings;
R₁ through R₁₈ are independently selected from the group consisting of hydrogen, hydroxyl, alkyl, hydroxyalkyl, alkyloxyalkyl, alkylcarboxyalkyl, terpenylcarboxyalkyl, terpenylcarbonyloxyalkyl, terpenylamidoalkyl, terpenyl-aminoalkyl, terpenyloxyoalkyl, alkylaminoalkyl, alkylaminoalkylamino, alkylaminoalkylaminoalkylamino, aminoalkyl, aryl, arylaminoalkyl, haloalkyl, alkenyl, alkynyl, oxo, linking group attached to a second steroid, aminoalkylurethanyl, aminoalkenylurethanyl, aminoalkynylurethanyl, aminoaryl-urethanyl, aminoalkyloxy, aminoalkylcarboxy, aminoalkyloxyalkyl, aminoalkyl-aminocarbonyl, aminoalkylcarboxamido, di(alkyl)aminoalkyl, alkylglyceride, H₂N—HC(Q₅)-(C═O)—O—, H₂N—HC(Q₅)-(C═O)—NH—, azidoalkyloxy, cyanoalkyloxy, P.G.-HN—HC(Q₅)-(C═O)—O—, guanidinoalkyloxy, quaternary ammonium alkylcarboxy, and guanidinoalkyl carboxy, where Q₅ is a side chain of any amino acid (including a side chain of glycine, i.e., H), and P.G. is an amino protecting group; and
R₅, R₈, R₉, R₁₀, R₁₃, R₁₄ and R₁₇ are independently deleted when one of rings A, B, C, or D is unsaturated so as to complete the valency of the carbon atom at that site,
provided that at least one, and sometimes two, three, or four, of R₁₋₄, R₆, R₇, R₁₁, R₁₂, R₁₅, R₁₆, R₁₇, and R₁₈ are independently selected from the group consisting of aminoalkyl, aminoalkyloxy, aminoalkylcarboxyalkyl, alkylaminoalkyl, alkylamino-alkylamino, alkylaminoalkylaminoalkylamino, aminoalkylcarboxy, aryl-aminoalkyl, aminoalkyloxyamino, alkylaminocarbonyl, aminoalkylaminocarbonyl, aminoalkyl-carboxyamido, di(alkyl)aminoalkyl, aminoalkylurethanyl, aminoalkenyl-urethanyl, aminoalkynylurethanyl, aminoarylurethanyl, H₂N—HC(Q₅)-C(O)—O—, H₂N—HC(Q₅)-C(O)—N(H)—, azidoalkyloxy, cyanoalkyloxy, P.G.-HN—HC(Q₅)-C(O)—O—, guanidinoalkyloxy, quaternary ammonium alkylcarboxy, and guanidino-alkylcarboxy.

In some embodiments, R₁ through R₄, R₆, R₇, R₁₁, R₁₂, R₁₅, R₁₆, and R₁₈ are independently selected from the group consisting of hydrogen, hydroxyl, substituted or unsubstituted (C₁-C₂₂)alkyl, substituted or unsubstituted (C₁-C₂₂)hydroxyalkyl, substituted or unsubstituted (C₁-C₂₂)alkyloxy-(C₁-C₂₂)alkyl, substituted or unsubstituted (C₁-C₂₂)alkylcarboxy-(C₁-C₂₂)alkyl, substituted or unsubstituted (C₅-C₂₅)terpenyl-carboxy-(C₁-C₂₂)alkyl, substituted or unsubstituted (C₅-C₂₅)terpenylcarbonyloxy-(C₁-C₂₂)alkyl, substituted or unsubstituted (C₅-C₂₅)terpenylcarboxamido-(C₁-C₂₂)alkyl, substituted or unsubstituted (C₅-C₂₅)terpenylamino-(C₁-C₂₂)alkyl, (C₅-C₂₅)terpenyloxyo-(C₁-C₂₂)alkyl, substituted or unsubstituted (C₁-C₂₂)alkylamino-(C₁-C₂₂)alkyl, substituted or unsubstituted (C₁-C₂₂)alkylamino-(C₁-C₂₂)alkylamino, substituted or unsubstituted (C₁-C₂₂)alkylamino-(C₁-C₂₂)alkylamino-(C₁-C₂₂)alkylamino, substituted or unsubsti-tuted (C₁-C₂₂)aminoalkyl, substituted or unsubstituted aryl, substituted or unsubstituted arylamino-(C₁-C₂₂)alkyl, substituted or unsubstituted (C₁-C₂₂)haloalkyl, substituted or unsubstituted (C₂-C₆)alkenyl, substituted or unsubstituted (C₂-C₆)alkynyl, oxo, linking group attached to a second steroid, substituted or unsubstituted (C₁-C₂₂)aminoalkylurethanyl, substituted or unsubstituted (C₂-C₂₂)aminoalkenylurethanyl, substituted or unsubstituted (C₂-C₂₂)aminoalkynylurethanyl, and substituted or unsubstituted aminoarylurethanyl, substituted or unsubstituted (C₁-C₂₂)aminoalkyloxy, substituted or unsubstituted (C₁-C₂₂)aminoalkylcarboxy, substituted or unsubstituted (C₁-C₂₂)aminoalkyloxy-(C₁-C₂₂)alkyl, substituted or unsubstituted (C₁-C₂₂)aminoalkyl-aminocarbonyl, substituted or unsubstituted (C₁-C₂₂)aminoalkylcarboxamido, substituted or unsubstituted di(C₁-C₂₂)alkylamino-(C₁-C₂₂)alkyl, H₂N—HC(Q₅)-(C═O)—O—, H₂N—HC(Q₅)-(C═O)—NH—, substituted or unsubstituted (C₁-C₂₂)azidoalkyloxy, substituted or unsubstituted (C₁-C₂₂)cyanoalkyloxy, P.G.-HN—HC(Q₅)-(C═O)—O—, substituted or unsubstituted (C₁-C₂₂)guanidinoalkyloxy, substituted or unsubstituted quaternary ammonium (C₁-C₂₂)alkylcarboxy, and substituted or unsubstituted (C₁-C₂₂)guanidinoalkyl carboxy, where Q₅ is a side chain of an amino acid (including a side chain of glycine, i.e., H), and P.G. is an amino protecting group; and
R₅, R₈, R₉, R₁₀, R₁₃, R₁₄ and R₁₇ are independently deleted when one of rings A, B, C, or D is unsaturated so as to complete the valency of the carbon atom at that site, or R₅, R₈, R₉, R₁₀, R₁₃, and R₁₄ are independently selected from the group consisting of hydrogen, hydroxyl, (C₁-C₂₂)alkyl, (C₁-C₂₂)hydroxyalkyl, (C₁-C₂₂)alkyloxy-(C₁-C₂₂)alkyl, (C₁-C₂₂) aminoalkyl, aryl, (C₁-C₂₂)haloalkyl, (C₂-C₆)alkenyl, (C₂-C₆)alkynyl, oxo, a linking group attached to a second steroid, (C₁-C₂₂)aminoalkyloxy, (C₁-C₂₂)aminoalkylcarboxy, (C₁-C₂₂)aminoalkylaminocarbonyl, di(C₁-C₂₂ alkyl)amino-(C₁-C₂₂)alkyl, H₂N—HC(Q₅)-C(O)—O—, H₂N—HC(Q₅)-C(O)—N(H)—, (C₁-C₂₂) azidoalkyloxy, (C₁-C₂₂) cyanoalkyloxy, P.G.-HN—HC(Q₅)-C(O)—O—, (C₁-C₂₂) guanidinoalkyloxy, and (C₁-C₂₂) guanidinoalkylcarboxy, where Q₅ is a side chain of an amino acid, and P.G. is an amino protecting group;
provided that at least two or three of R₁-4, R₆, R₇, R₁₁, R₁₂, R₁₅, R₁₆, R₁₇, and R₁₈ are independently selected from the group consisting of (C₁-C₂₂)aminoalkyl, (C₁-C₂₂)aminoalkyloxy, (C₁-C₂₂)alkylcarboxy-(C₁-C₂₂)alkyl, (C₁-C₂₂)alkylamino-(C₁-C₂₂)alkylamino, (C₁-C₂₂)alkylamino-(C₁-C₂₂)alkylamino-(C₁-C₂₂)alkylamino, (C₁-C₂₂)aminoalkylcarboxy, arylamino-(C₁-C₂₂)alkyl, (C₁-C₂₂)aminoalkyloxy (C₁-C₂₂)aminoalkylaminocarbonyl, (C₁-C₂₂)aminoalkyl-aminocarbonyl, (C₁-C₂₂)aminoalkylcarboxyamido, quaternary ammonium (C₁-C₂₂)alkylcarboxy, di(C₁-C₂₂ alkyl)amino-(C₁-C₂₂)alkyl, (C₁-C₂₂)aminoalkylurethanyl, (C₂-C₂₂)aminoalkenylurethanyl, (C₂-C₂₂)amino-alkynylurethanyl, aminoarylurethanyl, H₂N—HC(Q₅)-C(O)—O—, H₂N—HC(Q₅)-C(O)—N(H)—, (C₁-C₂₂) azidoalkyloxy, (C₁-C₂₂) cyanoalkyloxy, P.G.-HN—HC(Q₅)-C(O)—O—, (C₁-C₂₂) guanidinoalkyloxy, and (C₁-C₂₂) guanidinoalkylcarboxy.

In some embodiments, at least one of R₁-R₁₈, preferably R₁₈, can have the following bioresorbable mono- or diglyceride structure:

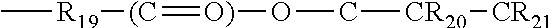

where R₁₉ is omitted or is selected from alkyl, alkenyl, alkynyl, and aryl, and R₂₀ and R₂₁ are independently selected from hydroxy and (C₂-C₂₂)alkylcarboxy, provided that at least one of R₂₀ or R₂₁ is (C₂-C₂₂)alkylcarboxy, the (C₂-C₂₂)alkylcarboxy preferably having an even number of carbons. The glyceride portion of foregoing structure forms bioresorbable glycerin and fatty acid(s) as degradation product (e.g., by hydrolysis of ester groups in the glyceride structure).

In some embodiments, R₁₈ can have the following bioresorbable mixed diglyceride structure:

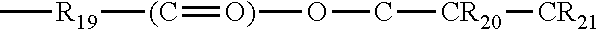

where R₁₉ is omitted or is selected from alkyl, alkenyl, alkynyl, and aryl, R₂₀ is a (C₂-C₂₂)alkylcarboxy, the (C₂-C₂₂)alkylcarboxy preferably having an even number of carbons, and R₂₁ can have the following aminoalkylcarboxy structure:

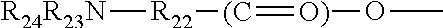

where R₂₂ is a substituted or unsubstituted alkyl and R₂₃ and R₂₄ are independently selected from hydrogen, alkyl, alkenyl, alkynyl, and aryl. R₂₂ is preferably an ester group of an amino acid, such as beta-alanine, which forms a bioresorbable amino acid (e.g., beta-alanine) as degradation product (e.g., by hydrolysis of the ester groups at the C₂₄). The glyceride portion forms bioresorbable glycerin, a fatty acid, and an amino acid as degradation products (e.g., by hydrolysis of ester groups in the glyceride structure).

In some embodiments, at least one of R₁-R₁₈, preferably at least one of R₃, R₇ and R₁₂, can have the following bioresorbable aminoalkylcarboxy or aminoalkylcarboxamido structure:

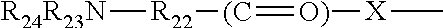

where R₂₂ is a substituted or unsubstituted alkyl, X is oxygen or nitrogen, and R₂₃ and R₂₄ are independently selected from hydrogen, alkyl, alkenyl, alkynyl, and aryl. At least one of R₃, R₇ and R₁₂, preferably two or three of R₃, R₇ and R₁₂, is/are an ester group of one or more amino acids, such as beta-alanine, which forms a bioresorbable amino acid (e.g., beta-alanine) as degradation product (e.g., by hydrolysis of the ester group(s) at the C₃, C₇ and/or C₁₂ position(s)). Alternatively, the aminoalkyl portion of at least one of R₃, R₇ and R₁₂ can be attached to one or more of the C₃, C₇ and/or C₁₂ positions of the sterol backbone (or elsewhere) by other linkages, such as amide or ether linkage.

In some embodiments, bioresorbable mono- and diglyceride CSA compounds can have a chiral center, such as in the glyceryl moiety, so as to form enantiomers that can be isolated rather than forming a racemic mixture. Unless otherwise specified, the examples of CSA compounds disclosed herein can be non-chiral, R- and S-enantiomers forming a racemic mixture, the R-enantiomer, or the S-enantiomer.

Non-limiting examples of bioresorbable monoglyceride CSA compounds that form bioresorbable degradation products by hydrolysis of ester groups are CSA-4108, CSA-4110 (racemic mixture), CSA-4110R (R-enantiomer), CSA-4110S (S-enantiomer), CSA-4112, CSA-4114, and salts thereof (see FIG. 1D). Non-limiting examples of bioresorbable diglyceride CSA compounds that form bioresorbable degradation products by hydrolysis of ester groups are CSA-4204, CSA-4206, CSA-4208, CSA-4210, and CSA-4310, and salts thereof (see FIG. 1D).

In some embodiments, R₁, R₂, R₄, R₅, R₆, R₈, R₉, R₁₀, R₁₁, R₁₃, R₁₄, R₁₅, R₁₆, and R₁₇ are independently selected from the group consisting of hydrogen and unsubstituted (C₁-C₆)alkyl.

In some embodiments, R₁, R₂, R₄, R₅, R₆, R₈, R₁₀, R₁₁, R₁₄, R₁₆, and R₁₇ are each hydrogen and R₉ and R₁₃ are each methyl.

In some embodiments, R₃, R₇, R₁₂, and R₁₈ are independently selected from the group consisting of hydrogen, (C₁-C₆)alkyl, (C₁-C₆)hydroxyalkyl, (C₁-C₁₆)alkyloxy-(C₁-C₅)alkyl, (C₁-C₁₆)alkylcarboxy-(C₁-C₅)alkyl, (C₁-C₁₆)alkylamino-(C₁-C₅)alkyl, (C₁-C₁₆)alkylamino-(C₁-C₅)alkylamino, (C₁-C₁₆)alkylamino-(C₁-C₁₆)alkylamino-(C₁-C₅)alkyl-amino, (C₅-C₂₅)terpenylcarboxy-(C₁-C₅)alkyl, (C₅-C₂₅)terpenylcarbonyloxy-(C₁-C₅)alkyl, (C₅-C₂₅)terpenylcarboxamido-(C₁-C₅)alkyl, (C₅-C₂₅)terpenylamino-(C₁-C₅)alkyl, (C₅-C₂₅)terpenyloxyo-(C₁-C₅)alkyl, (C₁-C₆)aminoalkylurethanyl, (C₂-C₆)aminoalkenylurethanyl, (C₂-C₆)aminoalkynylurethanyl, aminoarylurethanyl, (C₁-C₁₆)aminoalkyl, arylamino-(C₁-C₅)alkyl, (C₁-C₅)aminoalkyloxy, (C₁-C₁₆)aminoalkyl-oxy-(C₁-C₅)alkyl, (C₁-C₅)aminoalkylcarboxy, (C₁-C₅)aminoalkyl-aminocarbonyl, (C₁-C₅)aminoalkylcarbox-amido, di(C₁-C₅ alkyl)amino-(C₁-C₅)alkyl, (C₁-C₅)guanidino-alkyloxy, quaternary ammonium (C₁-C₁₆)alkylcarboxy, and unsubstituted (C₁-C₁₆) guanidinoalkylcarboxy.

In some embodiments, R₁, R₂, R₄, R₅, R₆, R₈, R₁₀, R₁₁, R₁₄, R₁₆, and R₁₇ are each hydrogen; and R₉ and R₁₃ are each methyl.

In some embodiments, R₃, R₇, R₁₂, and R₁₈ are independently selected from the group consisting of aminoalkyloxy, aminoalkylcarboxy, alkylaminoalkyl, alkoxycarbonylalkyl, alkylcarbonylalkyl, di(alkyl)aminoalkyl, alkylcarboxyalkyl, hydroxyalkyl, terpenylcarboxyalkyl, terpenylcarbonyloxyalkyl, terpenylcarboxamido-alkyl, terpenylamino-alkyl, terpenyloxyoalkyl, aminoalkylurethanyl, aminoalkenylurethanyl, aminoalkynyl-urethanyl, and aminoarylurethanyl.

In some embodiments, R₃, R₇, and R₁₂ are independently selected from the group consisting of aminoalkyloxy, aminoalkylcarboxy, aminoalkylcarboxamido, aminoalkylurethanyl, aminoalkenylurethanyl, aminoalkynylurethanyl, and aminoarylurethanyl.

In some embodiments, R₁₈ is selected from the group consisting of alkylaminoalkyl, alkoxycarbonylalkyl, alkylcarbonyloxyalkyl, alkylcarbonylalkyl, di(alkyl)aminoalkyl, alkylcarboxyalkyl, hydroxyalkyl, terpenylcarboxyalkyl, terpenylcarbonyloxyalkyl, terpenylcarboxamido-alkyl, terpenylaminoalkyl, terpenyloxyoalkyl, and alkylglyceride.

In some embodiments, one or more of rings A, B, C, and D is heterocyclic.

In some embodiments, rings A, B, C, and D are non-heterocyclic.

The compounds and compositions disclosed herein are optionally prepared as salts, which advantageously makes them cationic when one or more amine groups is/are protonated. “Salt” as used herein is a broad term, and is to be given its ordinary and customary meaning to a skilled artisan (and is not to be limited to a special or customized meaning), and refers without limitation to a salt of a compound. In embodiments, the salt is an acid addition salt of the compound. Salts can be obtained by reacting a compound with inorganic acids such as hydrohalic acid (e.g., hydrochloric acid or hydrobromic acid), sulfuric acid, nitric acid, phosphoric acid, and phosphonic acid. Salts can also be obtained by reacting a compound with an organic acid such as aliphatic or aromatic carboxylic or sulfonic acids, sulfinic acids, for example formic acid, acetic acid, propionic acid, glycolic acid, pyruvic acid, malonic acid, maleic acid, fumaric acid, trifluoroacetic acid, benzoic acid, cinnamic acid, mandelic acid, succinic acid, lactic acid, malic acid, tartaric acid, citric acid, ascorbic acid, nicotinic acid, methanesulfonic acid, ethanesulfonic acid, p-toluensulfonic acid, salicylic acid, stearic acid, muconic acid, butyric acid, phenylacetic acid, phenylbutyric acid, valproic acid, 1,2-ethanedisulfonic acid, 2-hydroxyethanesulfonic acid, benzenesulfonic acid, 2-naphthalenesulfonic acid, or 1,5-naphthalenedisulfonic acid (NDSA). Salts can also be obtained by reacting a compound with a base to form a salt such as an ammonium salt, an alkali metal salt, such as a lithium, sodium or a potassium salt, an alkaline earth metal salt, such as a calcium, magnesium or aluminum salt, a salt of organic bases such as dicyclohexylamine, N-methyl-D-glucamine, tris(hydroxymethyl)methylamine, C₁-C₇ alkylamine, cyclohexyl-amine, dicyclohexylamine, triethanolamine, ethylenediamine, ethanolamine, diethanolamine, triethanolamine, tromethamine, and salts with amino acids such as arginine and lysine; or a salt of an inorganic base, such as aluminum hydroxide, calcium hydroxide, potassium hydroxide, sodium carbonate, sodium hydroxide, or the like.

In embodiments, the salt is a hydrochloride salt. In embodiments, the salt is a mono-hydrochloride salt, a di-hydrochloride salt, a tri-hydrochloride salt, or a tetra-hydrochloride salt. Additional examples of salts include sulfuric acid addition salts, sulfonic acid addition salts, disulfonic acid addition salts, 1,5-naphthalenedisulfonic acid addition salts, sulfate salts, and bisulfate salts.

“R” groups such as, without limitation, R₁, R₂, R₃, R₄, R₅, R₆, R₇, R₈, R₉, R₁₀, R₁₁, R₁₂, R₁₃, R₁₄, R₁₅, R₁₆, R₁₇, and R₁₈, represent substituents that can be attached to the sterol backbone. Unless otherwise specified, an R group may be substituted or unsubstituted.

A “ring” can be heterocyclic or carbocyclic. “Saturated” means a ring in which each atom is either hydrogenated or substituted such that the valency of each atom is filled. “Unsaturated” means a ring where the valency of each atom of the ring may not be filled with hydrogen or other substituents. For example, adjacent carbon atoms in a fused ring can be double bound to each other. Unsaturation can also include deleting at least one of the following pairs and completing the valency of the ring carbon atoms at these deleted positions with a double bond, such as R₅ and R₉; R₈ and R₁₀; and R₁₃ and R₁₄.

Where a group is “substituted” it may be substituted with one, two, three or more of the indicated substituents, which may be the same or different, each replacing a hydrogen atom. If no substituents are indicated, the indicated “substituted” group may be substituted with one or more groups individually and independently selected from alkyl, alkenyl, alkynyl, cycloalkyl, cycloalkenyl, cycloalkynyl, acylalkyl, alkoxyalkyl, aminoalkyl, amino acid, aryl, heteroaryl, heteroalicyclyl, aralkyl, heteroaralkyl, (heteroalicyclyl)alkyl, hydroxy, protected hydroxyl, alkoxy, aryloxy, acyl, mercapto, alkylthio, arylthio, cyano, halogen (e.g., F, Cl, Br, and I), thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, oxo, silyl, sulfenyl, sulfinyl, sulfonyl, haloalkyl, haloalkoxy, trihalomethanesulfonyl, trihalomethanesulfonamido, an amino, a mono-substituted amino group and a di-substituted amino group, RₐO(CH₂)ₘO—, R_{b}(CH₂)ₙO—, R_{c}C(O)O(CH₂)ₚO—, and protected derivatives thereof. The substituent may be attached to the group at more than one attachment point. For example, an aryl group may be substituted with a heteroaryl group at two attachment points to form a fused multicyclic aromatic ring system. Biphenyl and naphthalene are two examples of an aryl group that is substituted with a second aryl group. A group that is not specifically labeled as substituted or unsubstituted may be considered to be either substituted or unsubstituted.

The terms “Cₐ” or “Cₐ to C_{b}” in which “a” and “b” are integers refer to the number of carbon atoms in an alkyl, alkenyl or alkynyl group, or the number of carbon atoms in the ring of a cycloalkyl, cycloalkenyl, cycloalkynyl, aryl, heteroaryl or heteroalicyclyl group. That is, the alkyl, alkenyl, alkynyl, ring of the cycloalkyl, ring of the cycloalkenyl, ring of the cycloalkynyl, ring of the aryl, ring of the heteroaryl or ring of the heteroalicyclyl can contain from “a” to “b”, inclusive, carbon atoms. Thus, for example, a “C₁ to C₄ alkyl” group refers to all alkyl groups having 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)—, (CH₃)₂CHCH₂— and (CH₃)₃C—. If no “a” and “b” are designated with regard to an alkyl, alkenyl, alkynyl, cycloalkyl cycloalkenyl, cycloalkynyl, aryl, heteroaryl or heteroalicyclyl group, the broadest range described in these definitions is to be assumed.

“Alkyl” means a straight or branched hydrocarbon chain that comprises a fully saturated (no double or triple bonds) hydrocarbon group. The alkyl group may have 1 to 25 carbon atoms (whenever it appears herein, a numerical range such as “1 to 25” refers to each integer in the given range; e.g., “1 to 25 carbon atoms” means that the alkyl group may consist of 1 carbon atom, 2 carbon atoms, 3 carbon atoms, etc., up to and including 25 carbon atoms, although the present definition also covers the occurrence of the term “alkyl” where no numerical range is designated). The alkyl group may also be a medium size alkyl having 1 to 15 carbon atoms. The alkyl group could also be a lower alkyl having 1 to 6 carbon atoms. The alkyl group of the compounds may be designated as “C₄” or “C₁-C₄ alkyl” or similar designations. By way of example only, “C₁-C₄ alkyl” indicates that there are one to four carbon atoms in the alkyl chain, i.e., the alkyl chain is selected from methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl and hexyl. The alkyl group may be substituted or unsubstituted.

“Alkenyl” means an alkyl group that contains in the straight or branched hydrocarbon chain one or more double bonds. The alkenyl group may have 2 to 25 carbon atoms (whenever it appears herein, a numerical range such as “2 to 25” refers to each integer in the given range; e.g., “2 to 25 carbon atoms” means that the alkenyl group may consist of 2, 3, or 4 carbon atoms, etc., up to and including 25 carbon atoms, although the present definition also covers the occurrence of the term “alkenyl” where no numerical range is designated). The alkenyl group may also be a medium size alkenyl having 2 to 15 carbon atoms. The alkenyl group could also be a lower alkenyl having 1 to 6 carbon atoms. The alkenyl group of the compounds may be designated as “C₄” or “C₂-C₄ alkenyl” or similar designations. An alkenyl group may be unsubstituted or substituted.

“Alkynyl” means an alkyl group that contains in the straight or branched hydrocarbon chain one or more triple bonds. The alkynyl group may have 2 to 25 carbon atoms (whenever it appears herein, a numerical range such as “2 to 25” refers to each integer in the given range; e.g., “2 to 25 carbon atoms” means that the alkynyl group may consist of 2, 3, or 4 carbon atoms, etc., up to and including 25 carbon atoms, although the present definition also covers the occurrence of the term “alkynyl” where no numerical range is designated). The alkynyl group may also be a medium size alkynyl having 2 to 15 carbon atoms. The alkynyl group could also be a lower alkynyl having 2 to 6 carbon atoms. The alkynyl group of the compounds may be designated as “C₄” or “C₂-C₄ alkynyl” or similar designations. An alkynyl group may be unsubstituted or substituted.

“Aryl” means a carbocyclic (all carbon) monocyclic or multicyclic aromatic ring system (including fused ring systems where two carbocyclic rings share a chemical bond) that has a fully delocalized pi-electron system throughout all the rings. The number of carbon atoms in an aryl group can vary. For example, the aryl group can be a C₆-C₁₄ aryl group, a C₆-C₁₀ aryl group, or a C₆ aryl group (although the definition of C₆-C₁₀ aryl covers the occurrence of “aryl” when no numerical range is designated). Examples of aryl groups include, but are not limited to, benzene, naphthalene and azulene. An aryl group may be substituted or unsubstituted.

“Aralkyl” and “aryl(alkyl)” mean an aryl group connected, as a substituent, via a lower alkylene group. The aralkyl group may have 6 to 20 carbon atoms (whenever it appears herein, a numerical range such as “6 to 20” refers to each integer in the given range; e.g., “6 to 20 carbon atoms” means that the aralkyl group may consist of 6 carbon atom, 7 carbon atoms, 8 carbon atoms, etc., up to and including 20 carbon atoms, although the present definition also covers the occurrence of the term “aralkyl” where no numerical range is designated). The lower alkylene and aryl group of an aralkyl may be substituted or unsubstituted. Examples include but are not limited to benzyl, 2-phenylalkyl, 3-phenylalkyl, and naphthylalkyl.

“Lower alkylene groups” mean a C₁-C₂₅ straight-chained alkyl tethering groups, such as —CH₂— tethering groups, forming bonds to connect molecular fragments via their terminal carbon atoms. Examples include but are not limited to methylene (—CH₂—), ethylene (—CH₂CH₂—), propylene (—CH₂CH₂CH₂—), and butylene (—CH₂CH₂CH₂CH₂—). A lower alkylene group can be substituted by replacing one or more hydrogen of the lower alkylene group with a substituent(s) listed under the definition of “substituted.”

“Cycloalkyl” means a completely saturated (no double or triple bonds) mono- or multi-cyclic hydrocarbon ring system. When composed of two or more rings, the rings may be joined together in a fused fashion. Cycloalkyl groups can contain 3 to 10 atoms in the ring(s) or 3 to 8 atoms in the ring(s). A cycloalkyl group may be unsubstituted or substituted. Typical cycloalkyl groups include, but are in no way limited to, cyclopropyl, cyclobutyl, cyclopentyl, cyclohexyl, cycloheptyl and cyclooctyl.

“Cycloalkenyl” means a mono- or multi-cyclic hydrocarbon ring system that contains one or more double bonds in at least one ring; although, if there is more than one, the double bonds cannot form a fully delocalized pi-electron system throughout all the rings (otherwise the group would be “aryl,” as defined herein). When composed of two or more rings, the rings may be connected together in a fused fashion. A cycloalkenyl group may be unsubstituted or substituted.

“Cycloalkynyl” means a mono- or multi-cyclic hydrocarbon ring system that contains one or more triple bonds in at least one ring. If there is more than one triple bond, the triple bonds cannot form a fully delocalized pi-electron system throughout all the rings. When composed of two or more rings, the rings may be joined together in a fused fashion. A cycloalkynyl group may be unsubstituted or substituted.

“Alkoxy” or “alkyloxy” mean the formula —OR wherein R is an alkyl, an alkenyl, an alkynyl, a cycloalkyl, a cycloalkenyl or a cycloalkynyl as defined above. Examples of alkoxys are methoxy, ethoxy, n-propoxy, 1-methylethoxy (isopropoxy), n-butoxy, iso-butoxy, sec-butoxy and tert-butoxy. An alkoxy may be substituted or unsubstituted.

“Acyl” means a hydrogen, alkyl, alkenyl, alkynyl, aryl, or heteroaryl connected, as substituents, via a carbonyl group, such as —(C═O)—R. Examples include formyl, acetyl, propanoyl, benzoyl, and acryl. An acyl may be substituted or unsubstituted.

“Alkoxyalkyl” or “alkyloxyalkyl” mean an alkoxy group connected, as a substituent, via a lower alkylene group. Examples include alkyl-O-alkyl- and alkoxy-alkyl- with the terms alkyl and alkoxy defined herein.

“Hydroxyalkyl” means an alkyl group in which one or more of the hydrogen atoms are replaced by a hydroxy group. Exemplary hydroxyalkyl groups include but are not limited to, 2-hydroxyethyl, 3-hydroxypropyl, 2-hydroxypropyl, and 2,2-dihydroxyethyl. A hydroxyalkyl may be substituted or unsubstituted.

“Haloalkyl” means an alkyl group in which one or more of the hydrogen atoms are replaced by a halogen (e.g., mono-haloalkyl, di-haloalkyl and tri-haloalkyl). Examples include chloromethyl, fluoromethyl, difluoromethyl, trifluoromethyl and 1-chloro-2-fluoromethyl, 2-fluoroisobutyl. A haloalkyl may be substituted or unsubstituted.

“Amino” means “—NH₂”.

“Hydroxy” means “—OH”.

“Cyano” means “—CN”.

“Carbonyl” or “oxo” mean “—C═O”.

“Azido” means “—N₃”.

“Aminoalkyl” means an amino group connected, as a substituent, via a lower alkylene group. Examples include H₂N-alkyl- with the term alkyl defined herein.

“Alkylcarboxyalkyl” means an alkyl group connected, as a substituent, to a carboxy group that is connected, as a substituent, to an alkyl group. Examples include alkyl-(C═O)—O-alkyl- and alkyl-O—(C═O)-alkyl- with the term alkyl as defined herein.

“Alkylaminoalkyl” means an alkyl group connected, as a substituent, to an amino group that is connected, as a substituent, to an alkyl group. Examples include alkyl-NH-alkyl- with the term alkyl as defined herein.

“Dialkylaminoalkyl” and “di(alkyl)aminoalkyl” mean two alkyl groups connected, each as a substituent, to an amino group that is connected, as a substituent, to an alkyl group. Examples include

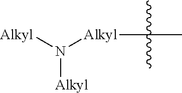

with the term alkyl as defined herein.

“Alkylaminoalkylamino” means an alkyl group connected, as a substituent, to an amino group that is connected, as a substituent, to an alkyl group that is connected, as a substituent, to an amino group. Examples include alkyl-NH-alkyl-NH— with the term alkyl as defined herein.

“Alkylaminoalkylaminoalkylamino” means an alkyl group connected, as a substituent, to an amino group that is connected, as a substituent, to an alkyl group that is connected, as a substituent, to an amino group that is connected, as a substituent, to an alkyl group. Examples include alkyl-NH-alkyl-NH-alkyl- with the term alkyl as defined herein.

“Arylaminoalkyl” means an aryl group connected, as a substituent, to an amino group that is connected, as a substituent, to an alkyl group. Examples include aryl-NH-alkyl- with the terms aryl and alkyl as defined herein.

“Aminoalkyloxy” means an amino group connected, as a substituent, to an alkyloxy group. Examples include H₂N-alkyl-O— and H₂N-alkoxy- with the terms alkyl and alkoxy as defined herein.

“Aminoalkyloxyalkyl” means an amino group connected, as a substituent, to an alkyloxy group connected, as a substituent, to an alkyl group. Examples include H₂N-alkyl-O-alkyl- and H₂N-alkoxy-alkyl- with the terms alkyl and alkoxy as defined herein.

“Aminoalkylcarboxy” means an amino group connected, as a substituent, to an alkyl group connected, as a substituent, to a carboxy group. Examples include H₂N-alkyl-(C═O)—O— and H₂N-alkyl-O—(C═O)— with the term alkyl as defined herein.

“Aminoalkylaminocarbonyl” means an amino group connected, as a substituent, to an alkyl group connected, as a substituent, to an amino group connected, as a substituent, to a carbonyl group. Examples include H₂N-alkyl-NH—(C═O)— with the term alkyl as defined herein.

“Aminoalkylcarboxamido” means an amino group connected, as a substituent, to an alkyl group connected, as a substituent, to a carbonyl group connected, as a substituent to an amino group. Examples include H₂N-alkyl-(C═O)—NH— and H₂N-alkyl-NH—(C═O)— with the term alkyl as defined herein.

“Azidoalkyloxy” means an azido group connected as a substituent, to an alkyloxy group. Examples include N₃-alkyl-O— and N₃-alkoxy- with the terms alkyl and alkoxy as defined herein.

“Cyanoalkyloxy” means a cyano group connected as a substituent, to an alkyloxy group. Examples include NC-alkyl-O— and NC-alkoxy- with the terms alkyl and alkoxy as defined herein.

“Sulfenyl” means “—SR” in which R can be hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. A sulfenyl may be substituted or unsubstituted.

“Sulfinyl” means “—(S═O)—R” in which R can be the same as defined with respect to sulfenyl. A sulfinyl may be substituted or unsubstituted.

“Sulfonyl” means “—(S═O)—OR” in which R can be the same as defined with respect to sulfenyl. A sulfonyl may be substituted or unsubstituted.

“O-carboxy” means “R—(C═O)—O—“in which R can be hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl, as defined herein. An O-carboxy may be substituted or unsubstituted.

“Ester” and “C-carboxy” mean “—(C═O)—OR” in which R can be the same as defined with respect to O-carboxy. An ester and C-carboxy may be substituted or unsubstituted.

“Thiocarbonyl” means “—(C═S)—R” in which R can be the same as defined with respect to O-carboxy. A thiocarbonyl may be substituted or unsubstituted.

“Trihalomethanesulfonyl” means “X₃CSO₂—” wherein X is a halogen.

“S-sulfonamido” means “—SO₂N(RARB)” in which RA and RB can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. An S-sulfonamido may be substituted or unsubstituted.

“N-sulfonamido” means “RSO₂N(RA)-” in which R and RA can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. An N-sulfonamido may be substituted or unsubstituted.

“O-carbamyl” and “urethanyl” mean “—O—(C═O)—N(RARB)” in which RA and RB can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. An O-carbamyl or urethanyl may be substituted or unsubstituted.

“N-carbamyl” means “RO—(C═O)—N(RA)-” in which R and RA can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. An N-carbamyl may be substituted or unsubstituted.

“O-thiocarbamyl” means “—O—(C═S)—N(RARB)” in which RA and RB can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. An O-thiocarbamyl may be substituted or unsubstituted.

“N-thiocarbamyl” means “RO—(C═S)—N(RA)-” in which R and RA can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. An N-thiocarbamyl may be substituted or unsubstituted.

“C-amido” means “—(C═O)—N(RARB)” in which RA and RB are independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. A C-amido may be substituted or unsubstituted.

“N-amido” means “R—(C═O)—N(RA)-” in which R and RA are independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. An N-amido may be substituted or unsubstituted.

“Guanidinoalkyloxy” means a guanidinyl group connected, as a substituent, to an alkyloxy group. Examples are

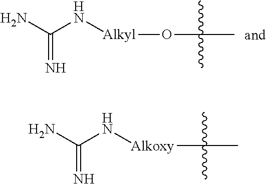

with the terms alkyl and alkoxy as defined herein.

“Guanidinoalkylcarboxy” means a guanidinyl group connected, as a substituent, to an alkyl group connected, as a substituent, to a carboxy group. Examples are

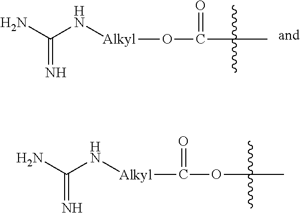

with the term alkyl as defined herein.

“Quaternary ammonium alkylcarboxy” means a quaternized amino group connected, as a substituent, to an alkyl group connected, as a substituent, to a carboxy group. Examples are

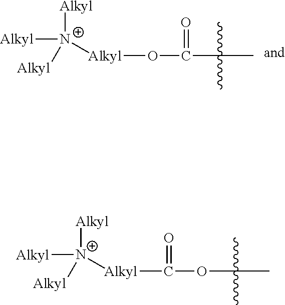

with the term alkyl as defined herein.

“Halogen atom” and “halogen” mean any one of the radio-stable atoms of column 7 of the Periodic Table of the Elements, such as, fluorine, chlorine, bromine and iodine.

Where the number of substituents is not specified (e.g. haloalkyl), there may be one or more substituents present. For example, “haloalkyl” may include one or more of the same or different halogens.

“Amino acid” means any amino acid (both standard and non-standard amino acids), including, but not limited to, α-amino acids, β-amino acids, γ-amino acids and δ-amino acids. Examples of suitable amino acids include, but are not limited to, alanine, asparagine, aspartate, cysteine, glutamate, glutamine, glycine, proline, serine, tyrosine, arginine, histidine, isoleucine, leucine, lysine, methionine, phenylalanine, threonine, tryptophan and valine. Additional examples of suitable amino acids include, but are not limited to, ornithine, hypusine, 2-aminoisobutyric acid, dehydroalanine, γ-aminobutyric acid, citrulline, β-alanine, α-ethyl-glycine, α-propyl-glycine and norleucine.

A “linking group” is a divalent moiety used to link one steroid to another steroid. In embodiments, the linking group is used to link a first CSA with a second CSA (which may be the same or different). An example of a linking group is (C₁-C₁₀) alkyloxy-(C₁-C₁₀) alkyl.

“P.G.” or “protecting group” or “protecting groups” mean any atom or group of atoms that is added to a molecule in order to prevent existing groups in the molecule from undergoing unwanted chemical reactions. Examples of protecting group moieties are described in T. W. Greene and P. G. M. Wuts, Protective Groups in Organic Synthesis, 3. Ed. John Wiley & Sons, 1999, and in J. F. W. McOmie, Protective Groups in Organic Chemistry Plenum Press, 1973, both of which are hereby incorporated by reference for the limited purpose of disclosing suitable protecting groups. The protecting group moiety may be chosen in such a way, that they are stable to certain reaction conditions and readily removed at a convenient stage using methodology known from the art. A non-limiting list of protecting groups include benzyl; substituted benzyl; alkylcarbonyls and alkoxycarbonyls (e.g., t-butoxycarbonyl (BOC), acetyl, or isobutyryl); arylalkylcarbonyls and arylalkoxycarbonyls (e.g., benzyloxycarbonyl); substituted methyl ether (e.g. methoxymethyl ether); substituted ethyl ether; substituted benzyl ether; tetrahydropyranyl ether; silyls (e.g., trimethylsilyl, triethylsilyl, triisopropylsilyl, t-butyldimethylsilyl, tri-iso-propylsilyloxymethyl, [2-(trimethylsilyl)ethoxy]methyl or t-butyldiphenylsilyl); esters (e.g. benzoate ester); carbonates (e.g. methoxymethylcarbonate); sulfonates (e.g. tosylate or mesylate); acyclic ketal (e.g. dimethyl acetal); cyclic ketals (e.g., 1,3-dioxane, 1,3-dioxolanes, and those described herein); acyclic acetal; cyclic acetal (e.g., those described herein); acyclic hemiacetal; cyclic hemiacetal; cyclic dithioketals (e.g., 1,3-dithiane or 1,3-dithiolane); orthoesters (e.g., those described herein) and triarylmethyl groups (e.g., trityl; monomethoxytrityl (MMTr); 4,4′-dimethoxytrityl (DMTr); 4,4′,4″-trimethoxytrityl (TMTr); and those described herein). Amino-protecting groups are known to those skilled in the art. In general, the species of protecting group is not critical, provided that it is stable to the conditions of any subsequent reaction(s) on other positions of the compound and can be removed at the appropriate point without adversely affecting the remainder of the molecule. In addition, a protecting group may be substituted for another after substantive synthetic transformations are complete. Clearly, where a compound differs from a compound disclosed herein only in that one or more protecting groups of the disclosed compound has been substituted with a different protecting group, that compound is within the disclosure.

### V. Additional Details of Pharmaceutical and Treatment Compositions

While CSA compounds described herein can be administered alone, it may be preferable to formulate the compounds as pharmaceutical or other treatment compositions (i.e., formulations), collectively referred to as “pharmaceutical compositions”. A pharmaceutical composition is any composition that may be administered in vitro or in vivo or both to a subject in order to treat or ameliorate a condition. In a preferred embodiment, a pharmaceutical composition may be administered in vivo. A subject may include one or more cells or tissues, or organisms. In exemplary embodiments, the subject is an animal. In embodiments, the animal is a mammal. The mammal may be a human or primate in some embodiments. A mammal includes any mammal, such as by way of non-limiting example, cattle, pigs, sheep, goats, horses, camels, buffalo, bison, cats, dogs, rats, mice, bats, pangolins, and humans.

“Pharmaceutically acceptable” and “physiologically acceptable” mean a biologically compatible formulation, gaseous, liquid or solid, or mixture thereof, which is suitable for one or more routes of administration, in vivo delivery, or contact. A formulation is compatible in that it does not destroy activity of an active ingredient therein (e.g., a CSA compound), or induce adverse side effects that far outweigh any prophylactic or therapeutic effect or benefit.

Pharmaceutical compositions may be formulated with a pharmaceutically acceptable excipient, such as a carrier, solvent, stabilizer, adjuvant, diluent, etc., depending upon the particular mode of administration and dosage form. The pharmaceutical compositions can be formulated to achieve a physiologically compatible pH, and may range from about 3 to 11, preferably about 3 to 7, depending on the formulation and route of administration. In alternative embodiments, the pH is adjusted to about 5 to 8. The pharmaceutical compositions may comprise a therapeutically or prophylactically effective amount of at least one compound as described herein, together with one or more pharmaceutically acceptable excipients.

The pharmaceutical composition may comprise a combination of compounds described herein and/or may include a second active ingredient useful in the treatment or prevention of bacterial infection (e.g., anti-bacterial or anti-microbial agents).

The composition can be formulated as a coating, such as on a medical device. In some embodiments, the coating is on a medical instrument.

Formulations for topical administration can in the form of a cream, liniment, salve, lotion, gel, liquid solution, spray, ointment, soap, or other such formulation readily administrable in a topical application. Topical administration can beneficially provide effective and long-lasting treatment for patients suffering from EB.

Formulations for parenteral or oral administration can be solids, liquid solutions, emulsions or suspensions. Inhalable formulations for pulmonary administration can be liquids or powders. A pharmaceutical composition can be formulated as a lyophilized solid that is reconstituted with a physiologically compatible solvent prior to administration. Alternative pharmaceutical compositions may be formulated as syrups, tablets, lozenges, mouthwashes, popping candies, sprays, creams, lotions, ointments, and gels, including spray gels.

Compositions may contain one or more excipients. Pharmaceutically acceptable excipients are determined in part by the particular composition being administered as well as by the particular method used to administer the composition. There exists a wide variety of suitable formulations of pharmaceutical compositions (see, e.g., Remington's Pharmaceutical Sciences).

Suitable excipients may be carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants such as ascorbic acid; chelating agents such as EDTA; carbohydrates such as dextrin, hydroxyalkylcellulose, hydroxyalkylmethylcellulose, stearic acid; liquids such as oils, water, saline, glycerol and ethanol; wetting or emulsifying agents; pH buffering substances; and the like. Liposomes are pharmaceutically acceptable excipients.

Pharmaceutical compositions may be formulated in any form suitable for the intended method of administration. When intended for oral use for example, tablets, troches, lozenges, aqueous or oil suspensions, non-aqueous solutions, dispersible powders or granules (including micronized particles or nanoparticles), emulsions, hard or soft capsules, syrups or elixirs may be prepared. Compositions intended for oral use may be prepared according to any method known to the art for the manufacture of pharmaceutical compositions, and such compositions may contain one or more agents including sweetening agents, flavoring agents, coloring agents and preserving agents, in order to provide a palatable preparation.

Pharmaceutically acceptable excipients particularly suitable for use in conjunction with tablets include, for example, inert diluents, such as celluloses, calcium or sodium carbonate, lactose, calcium or sodium phosphate; disintegrating agents, such as cross-linked povidone, maize starch, or alginic acid; binding agents, such as povidone, starch, gelatin or acacia; and lubricating agents, such as magnesium stearate, stearic acid or talc.

Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate, alone or with a wax, may be employed.

Formulations for oral use may be also presented as hard gelatin capsules where the active ingredient is mixed with an inert solid diluent, for example celluloses, lactose, calcium phosphate or kaolin, or as soft gelatin capsules wherein the active ingredient is mixed with non-aqueous or oil medium, such as glycerin, propylene glycol, polyethylene glycol, peanut oil, liquid paraffin or olive oil.

Pharmaceutical compositions can be formulated as a suspension comprising a CSA compound in admixture with at least one pharmaceutically acceptable excipient suitable for the manufacture of a suspension.

Pharmaceutical compositions can be formulated as dispersible powders and granules suitable for preparation of a suspension by the addition of suitable excipients.

Excipients suitable for use in connection with suspensions include suspending agents, such as sodium carboxymethylcellulose, methylcellulose, hydroxypropyl methylcellulose, sodium alginate, polyvinylpyrrolidone, gum tragacanth, gum acacia, dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxyethanol), a condensation product of ethylene oxide with a partial ester derived from a fatty acid and a hexitol anhydride (e.g., polyoxyethylene sorbitan monooleate); polysaccharides and polysaccharide-like compounds (e.g. dextran sulfate); glycoaminoglycans and glycosaminoglycan-like compounds (e.g., hyaluronic acid); and thickening agents, such as carbomer, beeswax, hard paraffin or cetyl alcohol. The suspensions may also contain one or more preservatives such as acetic acid, methyl and/or n-propyl p-hydroxy-benzoate; one or more coloring agents; one or more flavoring agents; and one or more sweetening agents such as sucrose or saccharin.

Pharmaceutical compositions may be in the form of oil-in water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth; naturally occurring phosphatides, such as soybean lecithin, esters or partial esters derived from fatty acids; hexitol anhydrides, such as sorbitan monooleate; and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

Pharmaceutical compositions may be in the form of a sterile injectable preparation, such as a sterile injectable aqueous emulsion or oleaginous suspension. The emulsion or suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,2-propandiol.

Sterile injectable preparations may also be prepared as a lyophilized powder. Among the acceptable vehicles and solvents that may be employed are water, Ringer's solution, and isotonic sodium chloride solution. In addition, sterile fixed oils may be employed as a solvent or suspending medium. For this purpose, any bland fixed oil may be employed including synthetic mono- or diglycerides. In addition, fatty acids such as oleic acid may likewise be used in the preparation of injectables.

To obtain a stable water-soluble dose form of a pharmaceutical composition, a pharmaceutically acceptable salt of a compound described herein may be dissolved in an aqueous solution of an organic or inorganic acid, such as 0.3 M solution of succinic acid, or more preferably, citric acid. If a soluble salt form is not available, the compound may be dissolved in a suitable co-solvent or combination of co-solvents. Examples of suitable co-solvents include alcohol, propylene glycol, polyethylene glycol 300, polysorbate 80, glycerin and the like in concentrations ranging from about 0 to 60% of the total volume. In one embodiment, the active compound is dissolved in DMSO and diluted with water.

Pharmaceutical composition may also be in the form of a solution of a salt form of the active ingredient in an appropriate aqueous vehicle, such as water or isotonic saline or dextrose solution. Also contemplated are compounds which have been modified by substitutions or additions of chemical or biochemical moieties which make them more suitable for delivery (e.g., increase solubility, bioactivity, palatability, decrease adverse reactions, etc.), for example by esterification, glycosylation, PEGylation, and complexation.

Many therapeutics have undesirably short half-lives and/or undesirable toxicity. Thus, the concept of improving half-life or toxicity is applicable to various treatments and fields. Pharmaceutical compositions can be prepared, however, by complexing the therapeutic with a biochemical moiety to improve such undesirable properties. Proteins are a particular biochemical moiety that may be complexed with a CSA for administration in a wide variety of applications. In some embodiments, one or more CSAs are complexed with a protein. In some embodiments, one or more CSAs are complexed with a protein to increase the CSA's half-life. In other embodiments, one or more CSAs are complexed with a protein to decrease the CSA's toxicity. Albumin is a particularly preferred protein for complexation with a CSA. In some embodiments, the albumin is fat-free albumin.

With respect to the CSA therapeutic, the biochemical moiety for complexation can be added to the pharmaceutical composition as 0.25, 0.5, 0.75, 1, 1.5, 2, 2.5, 3, 3.5, 4, 4.5, 5, 10, 20, 50, or 100 weight equivalents, or a range bounded by any two of the aforementioned numbers, or about any of the numbers. In embodiments, the weight ratio of albumin to CSA is about 18:1 or less, such as about 9:1 or less. In embodiments, the CSA is coated with albumin.

Non-biochemical compounds can be added to the pharmaceutical compositions to reduce the toxicity of the therapeutic and/or improve the half-life. Suitable amounts and ratios of an additive that can reduce toxicity can be determined via a cellular assay. With respect to the CSA therapeutic, toxicity reducing compounds can be added to the pharmaceutical composition as 0.25, 0.5, 0.75, 1, 1.5, 2, 2.5, 3, 3.5, 4, 4.5, 5, 10, 20, 50, or 100 weight equivalents, or a range bounded by any two of the aforementioned numbers, or about any of the numbers. In embodiments, the toxicity reducing compound is a cocoamphodiacetate such as Miranol® (disodium cocoamphodiacetate). In embodiments, the toxicity reducing compound is an amphoteric surfactant. In embodiments, the toxicity reducing compound is a surfactant. In embodiments, the molar ratio of cocoamphodiacetate to CSA is between about 8:1 and 1:1, preferably about 4:1. In embodiments, the toxicity reducing compound is allantoin.

In embodiments, a CSA composition is prepared utilizing one or more surfactants. In specific embodiments, the CSA is complexed with one or more poloxamer surfactants. Poloxamer surfactants are nonionic triblock copolymers composed of a central hydrophobic chain of polyoxypropylene (poly(propylene oxide)) flanked by two hydrophilic chains of polyoxyethylene (poly(ethylene oxide)). In some embodiments, the poloxamer is a liquid, paste, or flake (solid). Examples of suitable poloxamers include those by the trade names Synperonics, Pluronics, or Kolliphor. In some embodiments, one or more of the poloxamer surfactant in the composition is a flake poloxamer. In embodiments, the one or more poloxamer surfactant in the composition has a molecular weight of about 3600 g/mol for the central hydrophobic chain of polyoxypropylene and has about 70% polyoxyethylene content. In embodiments, the ratio of the one or more poloxamer to CSA is between about 50 to 1; about 40 to 1; about 30 to 1; about 20 to 1; about 10 to 1; about 5 to 1; about 1 to 1; about 1 to 10; about 1 to 20; about 1 to 30; about 1 to 40; or about 1 to 50. In embodiments, the ratio of the one or more poloxamer to CSA is between 50 to 1; 40 to 1; 30 to 1; 20 to 1; 10 to 1; 5 to 1; 1 to 1; 1 to 10; 1 to 20; 1 to 30; 1 to 40; or 1 to 50. In embodiments, the ratio of the one or more poloxamer to CSA is between about 50 to 1 to about 1 to 50. In embodiments, the ratio of the one or more poloxamer to CSA is between about 30 to 1 to about 3 to 1. In some embodiments, the poloxamer is Pluronic F127.

The amount of poloxamer may be based upon a weight percentage of the composition. In embodiments, the amount of poloxamer is about 10%, 15%, 20%, 25%, 30%, 35%, 40%, about any of the aforementioned numbers, or a range bounded by any two of the aforementioned numbers or the formulation. In embodiments, the one or more poloxamer is between about 10% to about 40% by weight of a formulation administered to the patient. In some embodiments, the one or more poloxamer is between about 20% to about 30% by weight of the formulation. In embodiments, the formulation contains less than about 50%, 40%, 30%, 20%, 10%, 5%, or 1% of CSA. In embodiments, the formulation containes less than about 20% by weight of CSA. The above described poloxamer formulations are particularly suited for the methods of treatment, device coatings, preparation of unit dosage forms (i.e., solutions, mouthwashes, sprays, lozenges (e.g., solid, disintegrable, “pop rocks” (popping candy or lozenge), injectables), etc.

In embodiments, the compounds described herein may be formulated for oral administration in a lipid-based formulation suitable for low solubility compounds. Lipid-based formulations can generally enhance the oral bioavailability of such compounds.

A pharmaceutical composition may comprise a therapeutically or prophylactically effective amount of a compound described herein, together with at least one pharmaceutically acceptable excipient selected from the group consisting of medium chain fatty acids or propylene glycol esters thereof (e.g., propylene glycol esters of edible fatty acids such as caprylic and capric fatty acids) and pharmaceutically acceptable surfactants such as polyoxyl 40 hydrogenated castor oil.

In embodiments, cyclodextrins may be added as aqueous solubility enhancers. Preferred cyclodextrins include hydroxypropyl, hydroxyethyl, glucosyl, maltosyl and maltotriosyl derivatives of α-, β-, and γ-cyclodextrin. A particularly preferred cyclodextrin solubility enhancer is hydroxypropyl-o-cyclodextrin (BPBC), which may be added to any of the above-described compositions to further improve the aqueous solubility characteristics of the compounds of the embodiments. In one embodiment, the composition comprises about 0.1% to about 20% hydroxypropyl-o-cyclodextrin, more preferably about 1% to about 15% hydroxypropyl-o-cyclodextrin, and even more preferably from about 2.5% to about 10% hydroxypropyl-o-cyclodextrin. The amount of solubility enhancer employed will depend on the amount of the compound of the embodiments in the composition.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A method of therapeutically or prophylactically treating a human patient suffering from epidermolysis bullosa (EB), comprising:
providing a CSA treatment composition that includes one or more cationic steroidal antimicrobial (CSA) compounds and a carrier;
administering the CSA treatment composition to soft tissue of the human patient suffering from EB; and
the CSA treatment composition initiating and/or accelerating healing of soft tissue wounds caused by EB and/or preventing or mitigating formation of soft tissue wounds caused by EB in the human patient.

2. The method of claim 1, wherein the human patient is an infant or child.

3. The method of claim 1, wherein the CSA treatment composition is administered topically to skin and/or mucous membrane of the human patient.

4. The method of claim 1, wherein the carrier comprises one or more of water, alcohol, other organic solvent, or emulsion.

5. The method of claim 1, wherein the CSA treatment composition is provided in the form of a liquid oral composition, liniment, salve, lotion, ointment, cream, powder, soap, wash, or spray.

6. The method of claim 1, wherein the one or more CSA compounds each has a structure of Formula IV, or salt thereof: 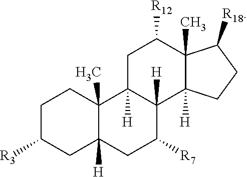
where,
R₃, R₇, and R₁₂ are independently selected from the group consisting of aminoalkyloxy, aminoalkylcarboxy, aminoalkylcarboxamido, aminoalkylurethanyl, aminoalkenylurethanyl, aminoalkynylurethanyl, and aminoarylurethanyl; and
R₁₈ is selected from the group consisting of alkylaminoalkyl, alkoxycarbonylalkyl, alkylcarbonyloxyalkyl, alkylcarbonylalkyl, di(alkyl)aminoalkyl, alkylcarboxyalkyl, hydroxyalkyl, terpenylcarboxyalkyl, terpenylcarbonyloxyalkyl, terpenylcarboxamido-alkyl, terpenylaminoalkyl, terpenyloxyoalkyl, and alkylglyceride.

7. The method of claim 1, wherein the one or more CSA compounds are selected from the group consisting of CSA-43, CSA-44, CSA-45, CSA-131, CSA-142, CSA-144, CSA-145, CSA-146, CSA-148, CSA-255, CSA 4108, CSA 4110, CSA 4110R, CSA 4110S, CSA 4112, CSA 4114, CSA 4204, CSA 4206, CSA 4208, CSA 4210, CSA 4310, and combinations thereof.

8. The method of claim 1, wherein the one or more CSA compounds initiate healing of soft tissue wounds caused by EB by triggering cell migration into wound beds and promoting neovascularization.

9. The method of claim 1, wherein the one or more CSA compounds provide anti-inflammatory activity in soft tissue wounds caused by EB.

10. The method of claim 9, wherein the one or more CSA compounds provide anti-inflammatory activity in soft tissue wounds caused by EB by sequestering bacterial endotoxins, reducing the release of some endogenous cytokines, and up-regulating the release of IL-8.

11. The method of claim 1, wherein the one or more CSA compounds provide antimicrobial activity in soft tissue wounds caused by EB.

12. The method of claim 11, wherein the one or more CSA compounds provide bactericidal activity against Gram-positive and Gram-negative bacteria, including providing bactericidal activity against drug-resistant bacteria and established biofilms that include bacteria.

13. The method of claim 11, wherein the one or more CSA compounds provide fungicidal activity against pathogenic fungi, including providing fungicidal activity against drug-resistant fungi and established biofilms that include fungi.

14. The method of claim 11, wherein the one or more CSA compounds provide virucidal activity, including inactivating lipid-enveloped viruses.

15. The method of claim 1, wherein the CSA treatment composition is effective in treating a human patient suffering from epidermolysis bullosa simplex (EBS).

16. The method of claim 1, wherein the CSA treatment composition is effective in treating a human patient suffering from dystrophic epidermolysis bullosa (DEB).

17. The method of claim 1, wherein the CSA treatment composition is effective in treating a human patient suffering from junctional epidermolysis bullosa (JEB).

18. The method of claim 1, wherein the CSA treatment composition is effective in treating a human patient suffering from EB.

19. A method of therapeutically treating a human patient suffering from epidermolysis bullosa (EB), comprising:
providing a CSA treatment composition that includes one or more cationic steroidal antimicrobial (CSA) compounds and a carrier;
administering the CSA treatment composition to soft tissue wounds caused by EB in the human patient; and
the CSA treatment composition initiating and/or accelerating healing of the soft tissue wounds caused by EB in the human patient.

20. A method of prophylactically treating a human patient suffering from epidermolysis bullosa (EB), comprising:
providing a CSA treatment composition that includes one or more cationic steroidal antimicrobial (CSA) compounds and a carrier;
administering the CSA treatment composition to soft tissue of the human patient suffering from EB; and
the CSA treatment composition preventing or mitigating formation of soft tissue wounds caused by EB in the human patient.
